(19) United States
(12) Patent Application Publication
(10) Pub. No.: US 2023/0201512 A1
(43) Pub. Date: Jun. 29, 2023

(54) METHODS AND APPARATUS FOR CONTROLLING OPERATIONS IN AN OXYGEN CONCENTRATOR

(71) Applicant: ResMed Asia Pte. Ltd., Singapore (SG)

(72) Inventors: Rex Dael NAVARRO, Singapore (SG); Jason Yuqian HUANG, Singapore (SG); Shayan MIARALIPOUR, Sydney (AU)

(73) Assignee: ResMed Asia Pte. Ltd., Singapore (SG)

(21) Appl. No.: 17/926,811

(22) PCT Filed: Jun. 15, 2021
(86) PCT No.: PCT/SG2021/050345

§ 371 Date: Nov. 21, 2022

Related U.S. Application Data
(60) Provisional application No. 62/705,499, filed on Jun. 30, 2020.

(51) Int. Cl. A61M 16/10; A61M 16/00; A61M 16/20; A61M 16/08; B01D 53/047

(52) U.S. Cl. CPC A61M 16/101; A61M 16/0063; A61M 16/201; A61M 16/022; A61M 16/105; A61M 16/085; B01D 53/047; A61M 2205/3365; B01D 2256/12; B01D 2257/102; B01D 2253/108; B01D 2259/40003; B01D 2259/40009; B01D 2259/402; B01D 2259/4541; B01D 2259/4533

(57) ABSTRACT

Methods and apparatus may implement controlled generation of oxygen enriched air in an oxygen concentrator while implementing control that reduces pneumatic imbalance between the concentrator's canisters, such as dynamic pressure imbalance or other pneumatic characteristic. One or more controllers may regulate operation of a compressor that feeds a pressurised air stream to the concentrator's canisters. This may regulate speed of the compressor to a speed set point for generating the pressurised stream. The regulating may involve generating a compressor control signal having a characteristic parameter such as a power parameter. The controller(s) may operate valve(s) in a cyclic pattern so as to produce oxygen enriched air in an accumulator. A cycle of the cyclic pattern may include a plurality of phases, where each of the plurality of phases has a duration. The controller(s) may then generate a dynamic adjustment to the duration(s) based on an evaluation of the characteristic parameter.

### CROSS REFERENCE TO RELATED APPLICATIONS

The present specification claims priority from U.S. Provisional Patent Application Ser. No. 62/705,499, filed on 30 Jun. 2020, the entire disclosure of which is hereby incorporated herein by reference.

### FIELD OF THE TECHNOLOGY

The present technology relates generally to methods and apparatus for treating respiratory disorders, such as those involving controlled pressure swing adsorption to generate oxygen enriched air. Such methodologies may be implemented in an oxygen concentrator. In some examples, the technology more specifically concerns methods and apparatus for control of operations of an oxygen concentrator such as for improving or maintaining operational efficiency. Such control of operations may be implemented to counteract imbalances that may develop in an oxygen concentrator over a period of use or extended use.

### DESCRIPTION OF THE RELATED ART

### Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Examples of respiratory disorders include respiratory failure, Obesity Hyperventilation Syndrome (OHS), Chronic Obstructive Pulmonary Disease (COPD), Neuromuscular Disease (NMD) and Chest wall disorders.

Respiratory failure is an umbrella term for respiratory disorders in which the lungs are unable to inspire sufficient oxygen or exhale sufficient CO₂ to meet the patient's needs. Respiratory failure may encompass some or all of the following disorders.

A patient with respiratory insufficiency (a form of respiratory failure) may experience abnormal shortness of breath on exercise.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors. Symptoms include: dyspnea on exertion, chronic cough and sputum production.

Neuromuscular Disease (NMD) is a broad term that encompasses many diseases and ailments that impair the functioning of the muscles either directly via intrinsic muscle pathology, or indirectly via nerve pathology. Some NMD patients are characterised by progressive muscular impairment leading to loss of ambulation, being wheelchair-bound, swallowing difficulties, respiratory muscle weakness and, eventually, death from respiratory failure. Neuromuscular disorders can be divided into rapidly progressive and slowly progressive. Rapidly progressive disorders are characterised by muscle impairment that worsens over months and results in death within a few years (e.g. Amyotrophic lateral sclerosis (ALS) and Duchenne muscular dystrophy (DMD) in teenagers). Variable or slowly progressive disorders are characterised by muscle impairment that worsens over years and only mildly reduces life expectancy (e.g. Limb girdle, Facioscapulohumeral and Myotonic muscular dystrophy). Symptoms of respiratory failure in NMD include: increasing generalised weakness, dysphagia, dyspnea on exertion and at rest, fatigue, sleepiness, morning headache, and difficulties with concentration and mood changes.

Chest wall disorders are a group of thoracic deformities that result in inefficient coupling between the respiratory muscles and the thoracic cage. The disorders are usually characterised by a restrictive defect and share the potential of long term hypercapnic respiratory failure. Scoliosis and/or kyphoscoliosis may cause severe respiratory failure. Symptoms of respiratory failure include: dyspnea on exertion, peripheral oedema, orthopnea, repeated chest infections, morning headaches, fatigue, poor sleep quality and loss of appetite.

### Respiratory Therapies

Various respiratory therapies, such as Non-invasive ventilation (NIV), Invasive ventilation (IV), and High Flow Therapy (HFT) have been used to treat one or more of the above respiratory disorders.

### Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Non-invasive ventilation (NIV) provides ventilatory support to a patient through the upper airways to assist the patient breathing and/or maintain adequate oxygen levels in the body by doing some or all of the work of breathing. The ventilatory support is provided via a non-invasive patient interface. NIV has been used to treat CSR and respiratory failure, in forms such as OHS, COPD, NMD and Chest Wall disorders. In some forms, the comfort and effectiveness of these therapies may be improved.

Invasive ventilation (IV) provides ventilatory support to patients that are no longer able to effectively breathe themselves and may be provided using a tracheostomy tube. In some forms, the comfort and effectiveness of these therapies may be improved.

### Flow Therapies

Not all respiratory therapies aim to deliver a prescribed therapeutic pressure. Some respiratory therapies aim to deliver a prescribed respiratory volume, by delivering an inspiratory flow rate profile over a targeted duration, possibly superimposed on a positive baseline pressure. In other cases, the interface to the patient's airways is ‘open’ (unsealed) and the respiratory therapy may only supplement the patient's own spontaneous breathing with a flow of conditioned or enriched air. In one example, High Flow therapy (HFT) is the provision of a continuous, heated, humidified flow of air to an entrance to the airway through an unsealed or open patient interface at a “treatment flow rate” that is held approximately constant throughout the respiratory cycle. The treatment flow rate is nominally set to exceed the patient's peak inspiratory flow rate. HFT has been used to treat OSA, CSR, respiratory failure, COPD, and other respiratory disorders. One mechanism of action is that the high flow rate of air at the airway entrance improves ventilation efficiency by flushing, or washing out, expired CO₂ from the patient's anatomical deadspace. Hence, HFT is thus sometimes referred to as a deadspace therapy (DST). Other benefits may include the elevated warmth and humidification (possibly of benefit in secretion management) and the potential for modest elevation of airway pressures. As an alternative to constant flow rate, the treatment flow rate may follow a profile that varies over the respiratory cycle.

Another form of flow therapy is long-term oxygen therapy (LTOT) or supplemental oxygen therapy. Doctors may prescribe a continuous flow of oxygen enriched air at a specified oxygen concentration (from 21%, the oxygen fraction in ambient air, to 100%) at a specified flow rate (e.g., 1 litre per minute (LPM), 2 LPM, 3 LPM, etc.) to be delivered to the patient's airway.

### Supplementary Oxygen

For certain patients, oxygen therapy may be combined with a respiratory pressure therapy or HFT by adding supplementary oxygen to the pressurised flow of air. When oxygen is added to respiratory pressure therapy, this is referred to as RPT with supplementary oxygen. When oxygen is added to HFT, the resulting therapy is referred to as HFT with supplementary oxygen.

### Respiratory Therapy Systems

These respiratory therapies may be provided by a respiratory therapy system or device. Such systems and devices may also be used to screen, diagnose, or monitor a condition without treating it.

A respiratory therapy system as described herein may comprise an oxygen source, an air circuit, and a patient interface.

### Oxygen Source

Experts in this field have recognized that exercise for respiratory failure patients provides long term benefits that slow the progression of the disease, improve quality of life and extend patient longevity. Most stationary forms of exercise like tread mills and stationary bicycles, however, are too strenuous for these patients. As a result, the need for mobility has long been recognized. Until recently, this mobility has been facilitated by the use of small compressed oxygen tanks or cylinders mounted on a cart with dolly wheels. The disadvantage of these tanks is that they contain a finite amount of oxygen and are heavy, weighing about 50 pounds when mounted.

Oxygen concentrators have been in use for about 50 years to supply oxygen for respiratory therapy. Oxygen concentrators may implement cyclic processes such as vacuum swing adsorption (VSA), pressure swing adsorption (PSA), or vacuum pressure swing adsorption (VPSA). For example, oxygen concentrators, e.g., POCs, may work based on depressurization (e.g., vacuum operation) and/or pressurization (e.g., compressor operation) in a swing adsorption process (e.g., Vacuum Swing Adsorption, Pressure Swing Adsorption or Vacuum Pressure Swing Adsorption, each of which are referred to herein as a “swing adsorption process”). Pressure swing adsorption may involve using one or more compressors to increase gas pressure inside one or more canisters that contains particles of a gas separation adsorbent. Such a canister when containing a mass of gas separation adsorbent such as a layer of gas separation adsorbent may be referred to as a sieve bed. As the pressure increases, certain molecules in the gas may become adsorbed onto the gas separation adsorbent. Removal of a portion of the gas in the canister under the pressurized conditions allows separation of the non-adsorbed molecules from the adsorbed molecules. The adsorbed molecules may then be desorbed by venting the canisters. Further details regarding oxygen concentrators may be found, for example, in U.S. Published Patent Application No. 2009-0065007, published Mar. 12, 2009, and entitled “Oxygen Concentrator Apparatus and Method”, which is incorporated herein by reference.

Ambient air usually includes approximately 78% nitrogen and 21% oxygen with the balance comprised of argon, carbon dioxide, water vapor and other trace gases. If a gas mixture such as air, for example, is fed under pressure through a canister containing a gas separation adsorbent that attracts nitrogen more strongly than it does oxygen, part or all of the nitrogen will be adsorbed by the adsorbent, and the gas coming out of the canister will be enriched in oxygen. When the adsorbent reaches the end of its capacity to adsorb nitrogen, the adsorbed nitrogen may be desorbed by venting. The canister is then ready for another cycle of producing oxygen enriched air. By alternating pressurization of the canisters in a two-canister system, one canister can be separating (or concentrating) oxygen while the other canister is being vented (resulting in a near-continuous separation of oxygen from the air). This alternation results in a near-continuous separation of the oxygen from the nitrogen. In this manner, oxygen enriched air can be accumulated, such as in a storage container or other pressurizable vessel or conduit coupled to the canisters, for a variety of uses including providing supplemental oxygen to users.

Vacuum swing adsorption (VSA) provides an alternative gas separation technique. VSA typically draws the gas through the separation process of the canisters using a vacuum such as a compressor configured to create a vacuum within the canisters. Vacuum Pressure Swing Adsorption (VPSA) may be understood to be a hybrid system using a combined vacuum and pressurization technique. For example, a VPSA system may pressurize the canisters for the separation process and also apply a vacuum for depressurizing the canisters.

Traditional oxygen concentrators have been bulky and heavy making ordinary ambulatory activities with them difficult and impractical. Recently, companies that manufacture large stationary oxygen concentrators began developing portable oxygen concentrators (POCs). The advantage of POCs is that they can produce a theoretically endless supply of oxygen and provide mobility for patients (users). In order to make these devices small for mobility, the various systems necessary for the production of oxygen enriched air are condensed. POCs seek to utilize their produced oxygen as efficiently as possible, in order to minimise weight, size, and power consumption. In some implementations, this may be achieved by delivering the oxygen enriched air as series of pulses, each pulse or “bolus” timed to coincide with the onset of inhalation. This therapy mode is known as pulsed oxygen delivery (POD) or demand mode, in contrast with traditional continuous flow delivery more suited to stationary oxygen concentrators. POD mode may be implemented with a conserver, which is essentially an active valve with a sensor to determine the onset of inhalation.

### Air Circuit

An air circuit is a conduit or a tube constructed and arranged to allow, in use, a flow of air to travel between two components of a respiratory therapy system such as the oxygen source and the patient interface. In some cases, there may be separate limbs of the air circuit for inhalation and exhalation. In other cases, a single limb air circuit is used for both inhalation and exhalation.

### Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH2O relative to ambient pressure. For other forms of therapy, such as the delivery of oxygen, the patient interface may not include a seal sufficient to facilitate delivery to the airways of a supply of gas at a positive pressure of about 10 cmH2O. For flow therapies such as nasal HFT, the patient interface is configured to insufflate the nares but specifically to avoid a complete seal. One example of such a patient interface is a nasal cannula.

### System Balance

An ideal PSA-based oxygen concentration system has symmetrical or balanced impedances such that there is a balanced mass flow of gas into, between, and out of the canisters. Optimal balance allows for optimal efficiency of the system i.e. optimal recovery of high purity oxygen with minimal power consumption. Unfortunately, a perfectly symmetrical PSA system is difficult to manufacture as component, material and assembly tolerances stack up. Operationally, imbalance may also develop over time in the form of system leaks as well as irregular output delivery of oxygen enriched air. If the canister imbalance gets too large or persists for too long, one canister may become exhausted well before the other, leading to a drop in purity or an overpressure that necessitates replacement of the adsorbent. A need therefore exists for control methods and apparatus that may be configured to counteract imbalance between canisters in a PSA system.

### SUMMARY OF THE TECHNOLOGY

Examples of the present technology may provide methods and apparatus for controlled operations of an oxygen concentrator, such as a portable oxygen concentrator.

In particular, the technology may provide methods and apparatus for a portable oxygen concentrator having a control mode to adjust the timings of one or more phases of the PSA cycle in order to reduce the imbalance of one or more pneumatic characteristics (e.g., pressure) between the pneumatic paths associated with the canisters. For example, the pressures in each canister may be estimated from a characteristic parameter of a control signal to a compressor that is being controlled to run at a regulated speed (e.g., a constant speed). Such a control signal may be evaluated to reduce pressure imbalance. In general, a characteristic parameter of the control signal can vary with the load on the compressor, and the varying characteristic parameter thereof can serve as an indication of the pneumatic characteristic of each canister.

For example, a variation in the control signal can be evaluated and taken as an indication of variation in the pressure in one or other of the canisters. Thus, the control signal characteristic parameter may be sampled at various points of each half of a PSA cycle to obtain a measure of imbalance in canister pressures. The measure of imbalance may be derived by comparison between one or more samples associated with a half cycle for one canister and one or more samples associated with a half cycle for another canister. The measure of imbalance may be then applied by the controller to set or adjust the PSA cycle phase durations, such as by making changes to the timing(s) of the valve(s) that are operated for each half cycle. For example, the measure of imbalance may be applied to a control modality, such as a proportional-integral controller or proportional-integral-derivative controller, so as to reduce the pressure imbalance between the canisters.

Some implementations of the present technology may include an oxygen concentrator. The oxygen concentrator may include a compressor configured to generate a pressurised air stream. The oxygen concentrator may include at least two canisters, each canister may include adsorbent material configured to preferentially adsorb a component gas from the pressurised air stream, thereby producing oxygen enriched air from the pressurised air stream. The oxygen concentrator may include one or more valves configured to selectively pneumatically couple the compressor to each canister so as to selectively feed the pressurised air stream to the canister. The one or more values may be configured to selectively vent each canister to atmosphere. The oxygen concentrator may include an accumulator pneumatically coupled to receive the produced oxygen enriched air. The oxygen concentrator may include one or more controllers operably coupled to the one or more valves and the compressor. The one or more controllers may be configured to regulate a speed of the compressor to a speed set point while generating the pressurised air stream, wherein the regulating may include generating a compressor control signal having a characteristic parameter. The one or more controllers may be configured to selectively operate the one or more valves, such as by generating one or more valve controls signals, in a cyclic pattern so as to produce oxygen enriched air in the accumulator. A cycle of the cyclic pattern may include a plurality of phases. Each of the plurality of phases may include a duration. The one or more controllers may be configured to generate a dynamic adjustment to one or more of the durations based on an evaluation of the characteristic parameter. The dynamic adjustment may reduce dynamic imbalance of a pneumatic characteristic between the canisters.

In some implementations, the one or more controllers may include an imbalance control system configured to generate the dynamic adjustment to the one or more durations. The imbalance control system may include a sampler. The sampler may be configured to sample or access one or more values of the characteristic parameter over a cycle. The sampler may be configured to compute a measure of imbalance based on the sampled values. The imbalance control system may include an imbalance controller configured to compute at least one phase duration adjustment from the measure of imbalance. The imbalance controller may be configured to compute the at least one phase duration adjustment based on a comparison between the measure of imbalance and an imbalance target value. The comparison may include a difference between the measure of imbalance and an imbalance target value. The imbalance controller may be a proportional-integral-derivative (PID) or proportional-integral (PI) controller.

In some implementations, the sampler may be configured to compute the measure of imbalance as a vector. The vector may include one or more of: (a) one or more differences between sample values at respective sampling points of consecutive half cycles; and (b) one or more ratios between sample values at respective sampling points of consecutive half cycles. The sampling points may coincide with phase transitions of the cycle. The sampler may be further configured to compute each sample value at a sampling point from a plurality of sample values leading up to and including the sampling point.

In some implementations, the evaluation may include a comparison between (a) a first sample value of the characteristic parameter, the first sample value being associated with at least one first phase for one of the at least two canisters, and (b) a second sample value of the characteristic parameter, the second sample value being associated with at least one second phase for another one of the at least two canisters, wherein the at least one first phase and the at least one second phase are corresponding phases. The comparison may include a difference between the first sample value and the second sample value. The comparison may include a ratio of the first sample value and the second sample value. The evaluation further may include determination of an error based on the comparison. The error may be determined from a target imbalance value.

In some implementations, the evaluation may include inputting the error to a proportional-integral-derivative (PID) or proportional-integral (PI) controller configured to generate the dynamic adjustment of the one or more of the durations. To regulate speed of the compressor to a speed set point, the one or more controllers may be configured to generate the compressor control signal based on a difference between (a) a measured speed signal generated by a speed sensor, and (b) the speed set point. The compressor control signal may be a pulse width modulation (PWM) waveform and the characteristic parameter may be a duty cycle of the PWM waveform.

Some implementations of the present technology may include a method of operating an oxygen concentration device. The method may include controlling, with one or more controllers, a compressor to generate a pressurised air stream to at least two canisters. Each canister may include adsorbent material configured to preferentially adsorb a component gas from the pressurised air stream, thereby producing oxygen enriched air from the pressurised air stream to an accumulator pneumatically coupled to receive the produced oxygen enriched air. The controlling of the compressor may include regulating a speed of the compressor to a speed set point. The regulating may include generating a compressor control signal having a characteristic parameter. The method may include controlling, with the one or more controllers, operation of one or more valves to selectively pneumatically couple the compressor to each canister so as to selectively feed the pressurised air stream to the canister. The method may include controlling, with the one or more controllers, operation of one or more valves to selectively vent each canister to atmosphere. The controlling operation of the one or more valves may include selectively operating the one or more valves in a cyclic pattern so as to produce the oxygen enriched air. A cycle of the cyclic pattern may include a plurality of phases. Each of the plurality of phases may include a duration. The method may include controlling, with the one or more controllers, generation of a dynamic adjustment to one or more of the durations based on an evaluation of the characteristic parameter. The dynamic adjustment may reduce dynamic imbalance of a pneumatic characteristic between the canisters.

In some implementations, to generate the dynamic adjustment to the one or more durations, the one or more controllers may (a) sample (e.g., access) one or more values of the characteristic parameter over a cycle, (b) compute a measure of imbalance based on the sampled values, and/or (c) compute at least one phase duration adjustment from the measure of imbalance. To compute the at least one phase duration adjustment, the one or more controllers may compare the measure of imbalance and an imbalance target value. To compare the measure of imbalance and an imbalance target value, the one or more controllers may compute a difference between the measure of imbalance and an imbalance target value. The one or more controllers may apply proportional-integral-derivative (PID) control or proportional-integral (PI) control to the computed difference. The one or more controllers may compute the measure of imbalance as a vector. The vector may include one or more of: (a) one or more differences between sample values at respective sampling points of consecutive half cycles; and (b) one or more ratios between sample values at respective sampling points of consecutive half cycles. The sampling points may coincide with phase transitions of the cycle. The one or more controllers may compute each sample value at a sampling point from a plurality of sample values leading up to and including the sampling point.

In some implementations, the evaluation may include comparing (a) a first sample value of the characteristic parameter, and (b) a second sample value of the characteristic parameter. The first sample value may be associated with at least one first phase for one of the at least two canisters. The second sample value may be associated with at least one second phase for another one of the at least two canisters. The at least one first phase and the at least one second phase may be corresponding phases. The comparing may include computing a difference between the first sample value and the second sample value. The comparing may include computing a ratio of the first sample value and the second sample value. The evaluation may further include determining an error based on the comparing. The error may include may be determined from a target imbalance value. The evaluation may include inputting the error to a proportional-integral-derivative (PID) or proportional-integral (PI) controller to generate the dynamic adjustment of the one or more of the durations.

In some implementations, the regulating of a speed of the compressor to a speed set point may include generating the compressor control signal based on a difference between (a) a measured speed signal generated by a speed sensor, and (b) the speed set point. The compressor control signal may be a pulse width modulation (PWM) waveform and the characteristic parameter may be a duty cycle of the PWM waveform.

Some implementations of the present technology may include apparatus. The apparatus may include means for controlling a compressor to generate a pressurised air stream to at least two canisters, each canister may include adsorbent material configured to preferentially adsorb a component gas from the pressurised air stream, thereby producing oxygen enriched air from the pressurised air stream to an accumulator pneumatically coupled to receive the produced oxygen enriched air; the controlling the compressor may include regulating a speed of the compressor to a speed set point, wherein the regulating may include generating a compressor control signal having a characteristic parameter.

The apparatus may include means for controlling operation of one or more valves to (a) selectively pneumatically couple the compressor to each canister so as to selectively feed the pressurised air stream to the canister, and (b) selectively vent each canister to atmosphere; wherein the controlling operation of the one or more valves may include selectively operating the one or more valves in a cyclic pattern so as to produce the oxygen enriched air, wherein a cycle of the cyclic pattern may include a plurality of phases, each of the plurality of phases may include a duration. The apparatus may include means for controlling generation of a dynamic adjustment to one or more of the durations based on an evaluation of the characteristic parameter, whereby the dynamic adjustment reduces dynamic imbalance of a pneumatic characteristic between the canisters.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### BRIEF DESCRIPTION OF THE DRAWINGS

Advantages of the present technology will become apparent to those skilled in the art with the benefit of the following detailed description of implementations and upon reference to the accompanying drawings in which similar reference numerals indicate similar components:

FIG. 1A depicts an oxygen concentrator in accordance with one form of the present technology.

FIG. 1B is a schematic diagram of the gas separation system of the oxygen concentrator of FIG. 1A.

FIG. 1C is a side view of the main components of the oxygen concentrator of FIG. 1A.

FIG. 1D is a perspective side view of a compression system of the oxygen concentrator of FIG. 1A.

FIG. 1E is a side view of a compression system that includes a heat exchange conduit.

FIG. 1F is a schematic diagram of example outlet components of the oxygen concentrator of FIG. 1A.

FIG. 1G depicts an outlet conduit for the oxygen concentrator of FIG. 1A.

FIG. 1H depicts an alternate outlet conduit for the oxygen concentrator of FIG. 1A.

FIG. 11 is a perspective view of a disassembled canister system for the oxygen concentrator of FIG. 1A.

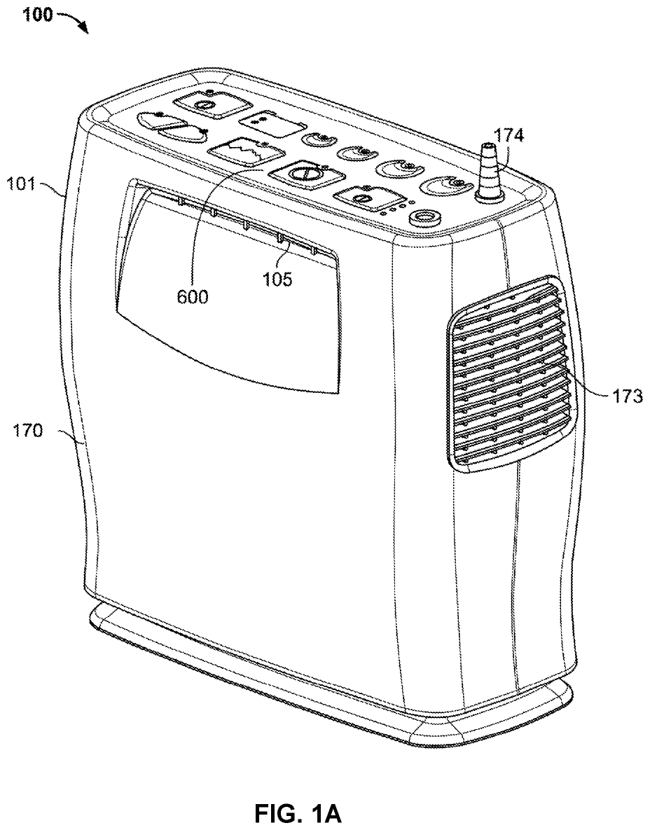

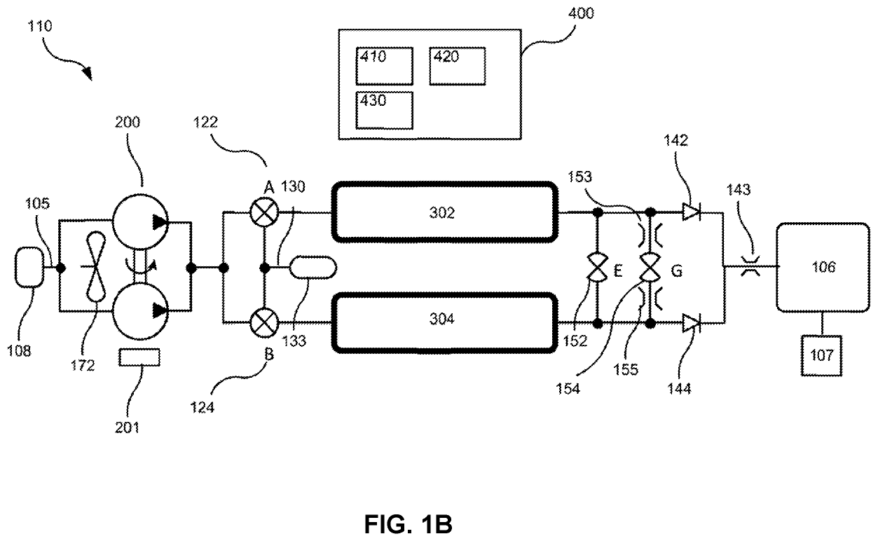

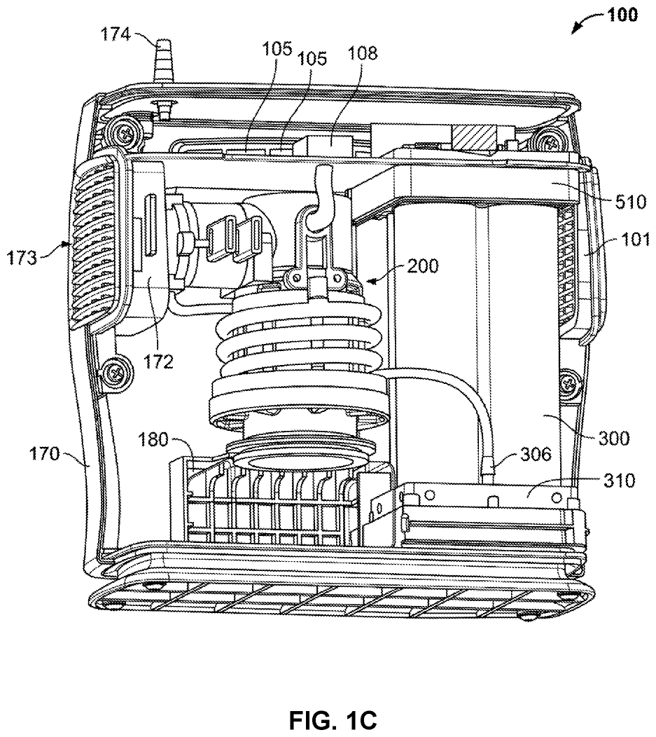

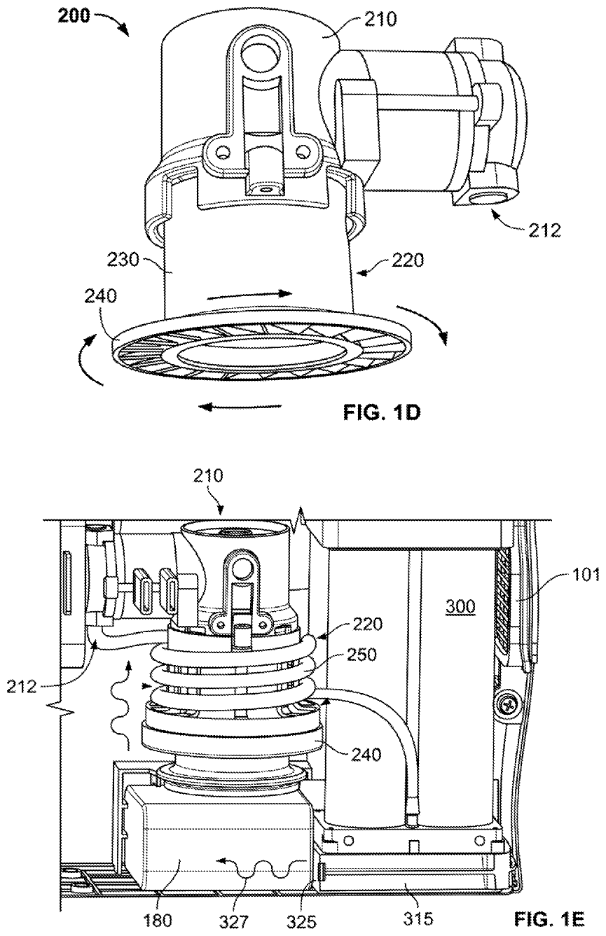

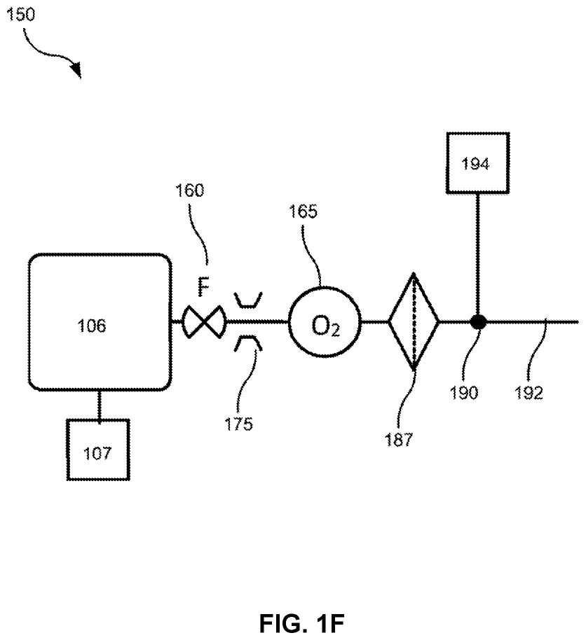

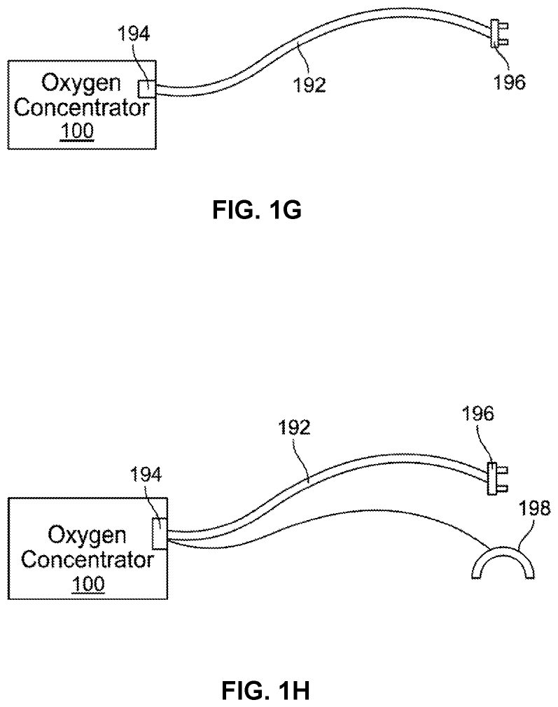

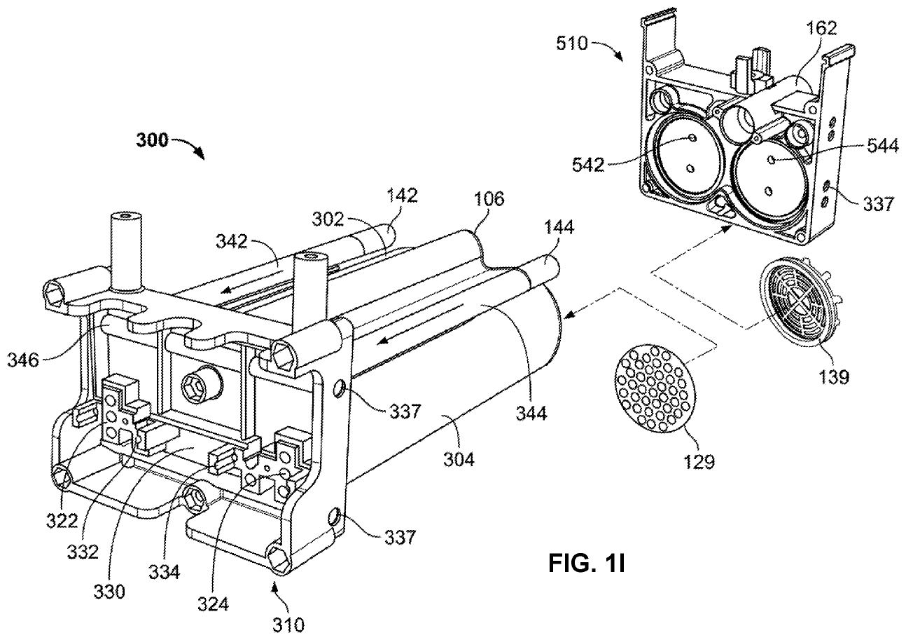

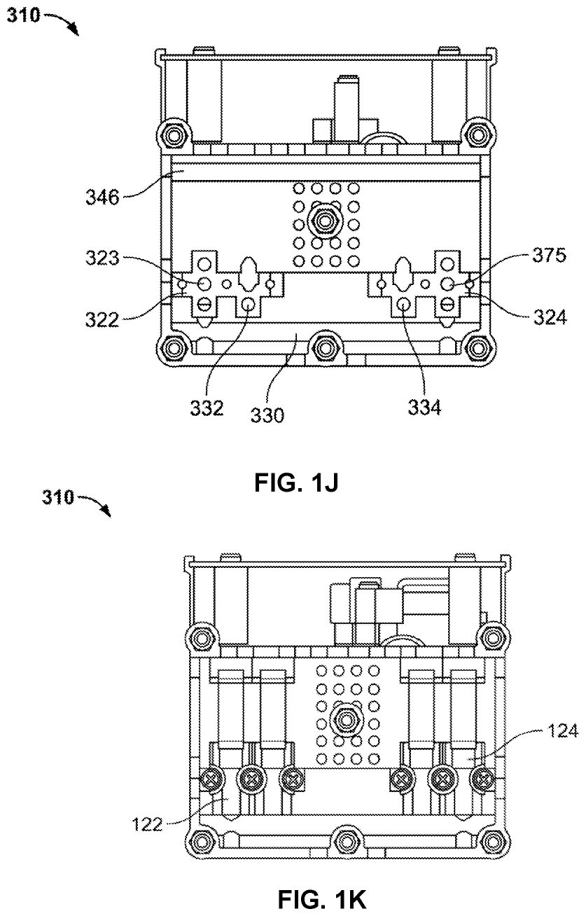

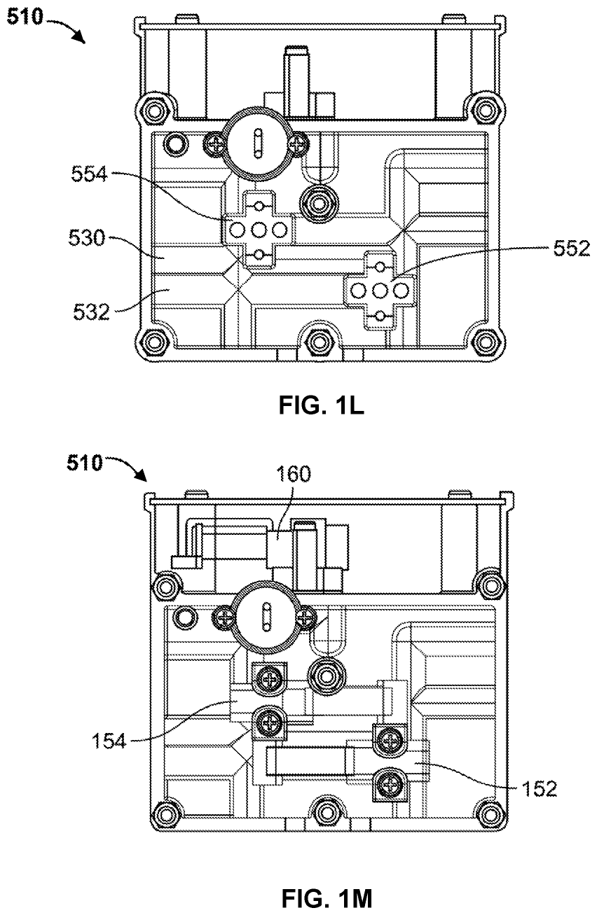

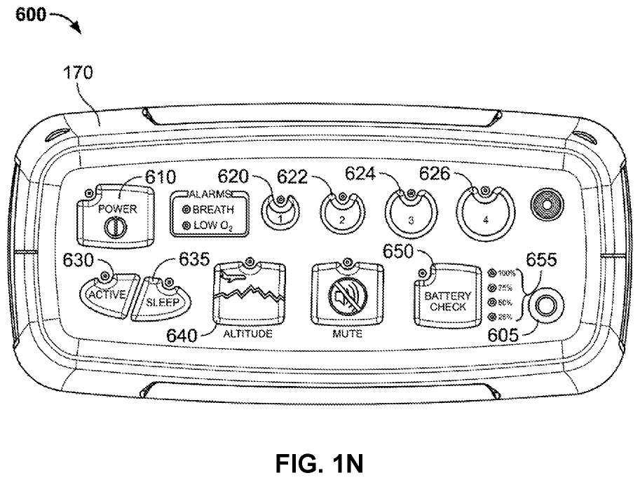

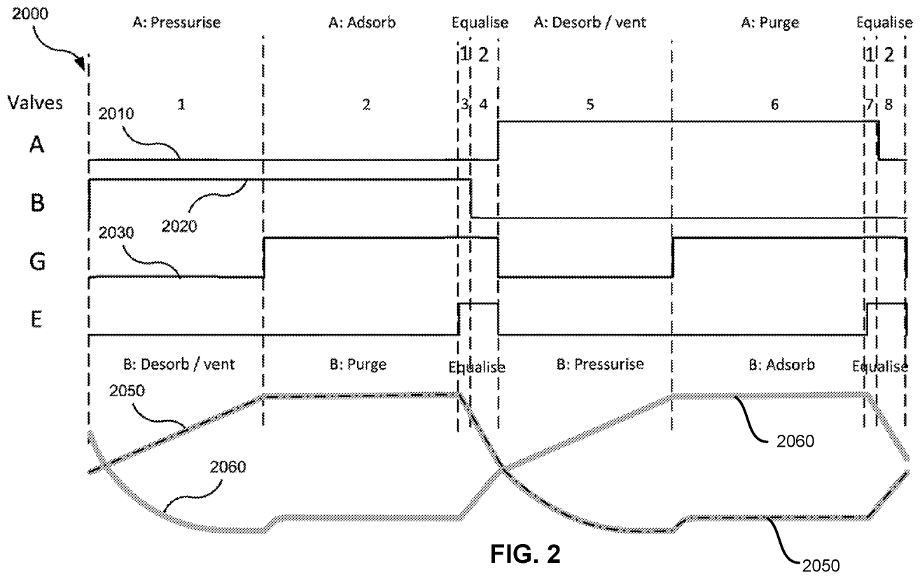

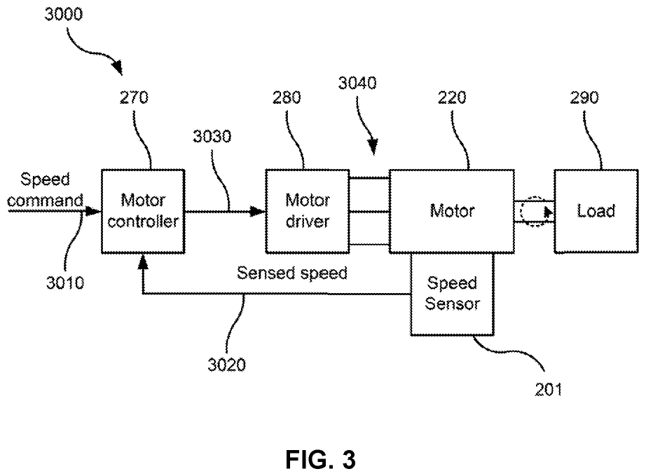

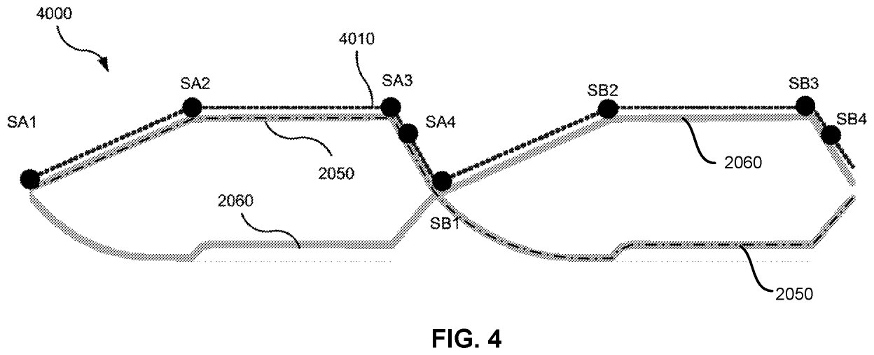

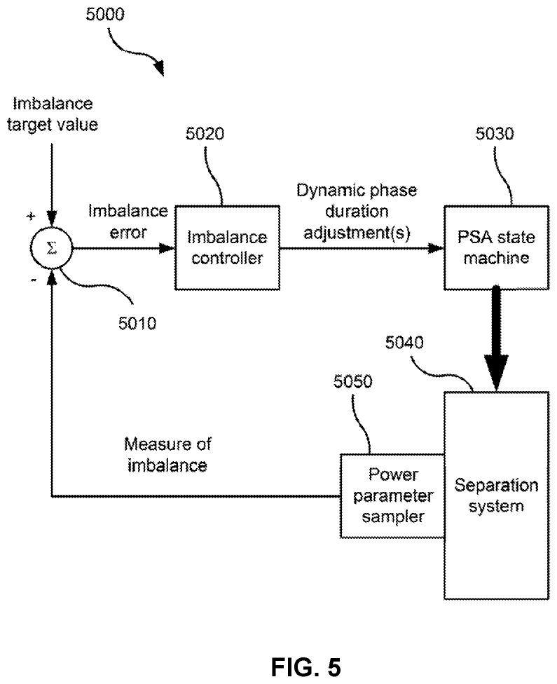

FIG. 1J is an end view of the canister system of FIG. 1I.

FIG. 1K is an assembled view of the canister system end depicted in FIG. 1J.

FIG. 1L is a view of an opposing end of the canister system of FIG. 1I to that depicted in FIGS. 1J and 1K.

FIG. 1M is an assembled view of the canister system end depicted in FIG. 1L.

FIG. 1N depicts an example control panel for the oxygen concentrator of FIG. 1A.

FIG. 2 is an illustration of one complete PSA cycle of a PSA process according to one implementation of the present technology.

FIG. 3 is a schematic diagram of a motor control circuit according to one implementation of the present technology.

FIG. 4 is a graph containing the canister pressure waveforms over a PSA cycle from FIG. 2, with a PWM duty cycle waveform superimposed.

FIG. 5 is a block diagram of an imbalance control system for a POC, such as the POC of FIG. 1A, according to one implementation of the present technology.

FIG. 6 contains four graphs showing the effect of a dynamic phase duration adjustment on the imbalance between the PWM duty cycle waveforms of consecutive PSA half cycles in a POC such as the POC of FIG. 1A.

### DETAILED DESCRIPTION OF THE IMPLEMENTATIONS

Aspects of the present technology are described in detail with reference to the drawing figures wherein like reference numerals identify similar or identical elements. It is to be understood that the disclosed implementation are merely examples of the technology, which may be implemented in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present technology in virtually any appropriately detailed structure.

FIGS. 1A to 1N illustrate an implementation of an oxygen concentrator 100. Oxygen concentrator 100 may concentrate oxygen within an air stream to provide oxygen enriched air to a user. Oxygen concentrator 100 may be a portable oxygen concentrator. For example, oxygen concentrator 100 may have a weight and size that allows the oxygen concentrator to be carried by hand and/or in a carrying case. In one implementation, oxygen concentrator 100 has a weight of less than about 20 pounds, less than about 15 pounds, less than about 10 pounds, or less than about 5 pounds. In an implementation, oxygen concentrator 100 has a volume of less than about 1000 cubic inches, less than about 750 cubic inches, less than about 500 cubic inches, less than about 250 cubic inches, or less than about 200 cubic inches.

As described herein, oxygen concentrator 100 uses a cyclic pressure swing adsorption (PSA) process to produce oxygen enriched air. However, in other implementations, oxygen concentrator 100 may be modified such that it uses a cyclic vacuum swing adsorption (VSA) process or a cyclic vacuum pressure swing adsorption (VPSA) process to produce oxygen enriched air.

### Outer Housing

FIG. 1A depicts an implementation of an outer housing 170 of an oxygen concentrator 100. In some implementations, outer housing 170 may be comprised of a light-weight plastic. Outer housing 170 includes compression system inlets 105, cooling system passive inlet 101 and outlet 173 at each end of outer housing 170, outlet port 174, and control panel 600. Inlet 101 and outlet 173 allow cooling air to enter the housing, flow through the housing, and exit the interior of housing 170 to aid in cooling of the oxygen concentrator 100. Compression system inlets 105 allow air to enter the compression system. Outlet port 174 is used to attach a conduit to provide oxygen enriched air produced by the oxygen concentrator 100 to a user.

### Gas Separation System

FIG. 1B illustrates a schematic diagram of a gas separation system 110 of an oxygen concentrator such as the oxygen concentrator 100, according to one implementation of the present technology. Separation system 110 may concentrate oxygen within an air stream to provide oxygen enriched air to an outlet system (described below).

Oxygen enriched air may be produced from ambient air by pressurising ambient air in canisters 302 and 304, which contain a gas separation adsorbent and are therefore referred to as sieve beds. Gas separation adsorbents useful in an oxygen concentrator are capable of separating at least nitrogen from an air stream to produce oxygen enriched air. Examples of gas separation adsorbents include molecular sieves that are capable of separating nitrogen from an air stream. Examples of adsorbents that may be used in an oxygen concentrator include, but are not limited to, zeolites (natural) or synthetic crystalline aluminosilicates that separate nitrogen from an air stream under elevated pressure. Examples of synthetic crystalline aluminosilicates that may be used include, but are not limited to: OXYSIV adsorbents available from UOP LLC, Des Plaines, IW; SYLOBEAD adsorbents available from W. R. Grace & Co, Columbia, Md.; SILIPORITE adsorbents available from CECA S. A. of Paris, France; ZEOCHEM adsorbents available from Zeochem AG, Uetikon, Switzerland; and AgLiLSX adsorbent available from Air Products and Chemicals, Inc., Allentown, Pa.

As shown in FIG. 1B, air may enter the separation system 110 through air inlet 105. Air may be drawn into air inlet 105 by compression system 200. Compression system 200 may draw in air from the surroundings of the oxygen concentrator and compress the air, forcing the compressed air into one or both canisters 302 and 304. In an implementation, an inlet muffler 108 may be coupled to air inlet 105 to reduce sound produced by air being pulled into the oxygen concentrator by compression system 200. In an implementation, inlet muffler 108 may reduce moisture as well as sound. For example, a water adsorbent material (such as a polymer water adsorbent material or a zeolite material) may be used to both adsorb water from the incoming air and to reduce the sound of the air passing into the air inlet 105. In an implementation, inlet muffler 108 may reduce contaminant particles (dust) and sound. For example, a dust filter may be used to both remove dust from the incoming air and to reduce the sound of the air passing into the air inlet 105.

Compression system 200 may include one or more compressors configured to compress air. Pressurized air, produced by compression system 200, may be fed into one or both of the canisters 302 and 304. In some implementations, the ambient air may be pressurized in the canisters to a target pressure approximately in a range of 13-25 pounds per square inch gauge (psig). Other target pressure values may also be used, depending on the type of gas separation adsorbent disposed in the canisters.

Coupled to each canister 302/304 are valves such as three-way inlet valves 122/124. As shown in FIG. 1B, inlet valve 122 (labelled A) is coupled to the “feed end” of canister 302 and inlet valve 124 (labelled B) is coupled to the “feed end” of canister 304. Inlet valves 122/124 are used to control the passage of air from compression system 200 to the respective canisters, and to vent exhaust gas from the respective canisters to atmosphere. In some implementations, inlet valves 122/124 may be silicon plunger solenoid valves. Other types of valves, however, may be used, such as poppet valves or piezoelectric valves. Plunger valves offer advantages over other kinds of valves by being quiet and having low slippage. In some implementations, one or both inlet valves 122/124 may be replaced by pairs of two-way valves that actuate in antiphase to emulate a three-way valve.

In some implementations, a two-step valve actuation voltage may be generated to control inlet valves 122/124. For example, a high voltage (e.g., 24 V) may be applied to an inlet valve to actuate the inlet valve. The voltage may then be reduced (e.g., to 7 V) to keep the inlet valve actuated. Using less voltage to keep a valve actuated may use less power. This reduction in voltage minimizes heat buildup and power consumption to extend run time from the power supply 180 (described below). When the power is cut off to an inlet valve 122/124, the valve de-actuates by spring action. In some implementations, the voltage may be applied as a function of time that is not necessarily a stepped response (e.g. a curved downward voltage between an initial 24 V and a final 7 V).

In an implementation, a controller 400 is electrically coupled to inlet valves 122 and 124 by an input/output interface. Controller 400 includes one or more processors 410 operable to execute program instructions stored in memory 420. The program instructions configure the controller to perform various predefined methods that are used to operate the oxygen concentrator, such as the methods described in more detail herein. The program instructions may include program instructions for generating control signals via the output interface to operate inlet valves 122 and 124 in a cyclic pattern to implement a cyclic PSA process as herein described. In some implementations, the voltages and the durations of the voltages used to open the inlet valves may be controlled by controller 400. The controller 400 may also include a transceiver 430 that may communicate with external devices to transmit data collected by the processor 410 or receive instructions from an external device for the processor 410.

Check valves 142 and 144 are coupled to the “product ends” of canisters 302 and 304, respectively. Check valves 142 and 144 may be one-way valves that are passively operated by the pressure differentials that occur as the canisters are pressurized and vented, or may be active valves. Check valves 142 and 144 are coupled to the canisters to allow oxygen enriched air produced during pressurization of each canister to flow out of the canister, and to inhibit back flow of oxygen enriched air or any other gases into the canister. In this manner, check valves 142 and 144 act as one-way valves allowing oxygen enriched air to exit the respective canisters during pressurization.

The term “check valve”, as used herein, refers to a valve that allows flow of a fluid (gas or liquid) in one direction and inhibits back flow of the fluid. The term “fluid” may include a gas or a mixture of gases (such as air). Examples of check valves that are suitable for use include, but are not limited to: a ball check valve; a diaphragm check valve; a butterfly check valve; a swing check valve; a duckbill valve; an umbrella valve; and a lift check valve. Under pressure, nitrogen molecules in the pressurized ambient air are adsorbed by the gas separation adsorbent in the pressurized canister. As the pressure increases, more nitrogen is adsorbed until the gas in the canister is enriched in oxygen. The nonadsorbed gas molecules (mainly oxygen) flow out of the pressurized canister when the pressure reaches a point sufficient to overcome the resistance of the check valve coupled to the canister. In one implementation, the pressure drop of the check valve in the forward direction is less than 1 psi. The break pressure in the reverse direction is greater than 100 psi. It should be understood, however, that modification of one or more components would alter the operating parameters of these valves. If the forward flow pressure is increased, there is, generally, a reduction in oxygen enriched air production. If the break pressure for reverse flow is reduced or set too low, there is, generally, a reduction in oxygen enriched air pressure.

In an implementation, pressurized air is sent into one of canisters 302 or 304 while the other canister is being vented. For example, during use, inlet valve 122 is de-actuated while inlet valve 124 is actuated. Pressurized air from compression system 200 is fed into canister 302 via the de-actuated inlet valve 122, while being inhibited from entering canister 304 by the actuated inlet valve 124. During pressurization of canister 302, actuated inlet valve 124 connects canister 304 to atmosphere to allow exhaust gas (mainly nitrogen) to vent from canister 304 to atmosphere through concentrator outlet 130. In an implementation, the exhaust gas may be directed through muffler 133 to reduce the noise produced by venting the exhaust gas from the canister. As exhaust gas is vented from canister 304, the pressure in the canister 304 drops, allowing the nitrogen to become desorbed from the gas separation adsorbent. The desorption of the nitrogen resets canister 304 to a state that allows renewed separation of nitrogen from a feed air stream. Muffler 133 may include open cell foam (or another material) to muffle the sound of the exhaust gas leaving the oxygen concentrator. In some implementations, the combined muffling components/techniques for the input of air and the output of oxygen enriched air may provide for oxygen concentrator operation at a sound level below 50 decibels.

After some time, the pressure in canister 302 is sufficient to open check valve 142. Oxygen enriched air produced in canister 302 passes through check valve 142 and flow restrictor 143 and, in one implementation, is collected in accumulator 106. The flow restrictor 143 controls the flow of oxygen enriched air to the accumulator 106. For example, when the accumulator 106 is depressurised upon bolus release (described below), if flow restrictor 143 is not present (and the intervening path has very low impedance), accumulator 106 draws gas at a high flow rate from the canister currently in pressurisation or adsorption. As a result, the pressure in the canister significantly drops, which tends to draw un-enriched air to the accumulator 106, thereby reducing the oxygen purity. Additionally, gas exchange between sieve beds via the E- and G-valves 152 and 154 to maintain high oxygen purity at the product end of the canisters will be vastly affected causing disruption in the overall PSA cycle. The presence of the flow restrictor 143 helps to replace released oxygen enriched air at an optimal rate and damp the above detrimental effects.

After some further time, the gas separation adsorbent in canister 302 becomes saturated with nitrogen and is unable to separate significant amounts of nitrogen from incoming air. This point is usually reached after a predetermined time of oxygen enriched air production. In the implementation described above, when the gas separation adsorbent in canister 302 reaches this saturation point, a two-way valve 152 (labelled E) is actuated, which directly connects canister 302 to 304 at their product ends. This causes the pressure in canister 302 to fall rapidly while the pressure in canister 304 rises equally rapidly towards equilibrium with canister 302. Inlet valve 124 is then de-actuated, connecting compression system 200 to canister 304 to assist with this equalisation of pressures from the feed end. Once the pressures in the canisters are equalised, which occurs after a predetermined time, valve 152 is de-actuated to isolate the canisters once again, and inlet valve 122 is actuated, stopping the feed of compressed air to canister 302 and connecting canister 302 to atmosphere to allow venting of exhaust gas. While canister 302 is being vented, canister 304 is pressurized to produce oxygen enriched air in the same manner described above. Pressurization of canister 304 is achieved through de-actuated inlet valve 124. After a time, the oxygen enriched air exits canister 304 through check valve 144.

During venting of the canisters, it is advantageous that at least a majority of the nitrogen is removed. In an implementation, at least about 50%, at least about 60%, at least about 70%, at least about 80%, at least about 90%, at least about 95%, at least about 98%, or substantially all of the nitrogen in a canister is removed before the canister is re-used to separate nitrogen from air.

In some implementations, nitrogen removal may be assisted using an oxygen enriched air stream that is introduced into the canister from the other canister or stored oxygen enriched air. In an exemplary implementation, a portion of the oxygen enriched air may be transferred from canister 302 to canister 304 when canister 304 is being vented of exhaust gas. Transfer of oxygen enriched air from canister 302 to canister 304 during venting of canister 304 helps to desorb nitrogen from the adsorbent by lowering the partial pressure of nitrogen adjacent the adsorbent. The flow of oxygen enriched air also helps to purge desorbed nitrogen (and other gases) from the canister. In an implementation, oxygen enriched air may travel through flow restrictors 153 and 155 between the two canisters. Flow restrictors 153 and 155 may be 0.013D flow restrictors. Other flow restrictor types and sizes are also contemplated and may be used depending on the specific configuration and tubing used to couple the canisters. In some implementations, the flow restrictors 153 and 155 may be press fit flow restrictors that restrict air flow by introducing a narrower diameter in their respective conduits. In some implementations, the press fit flow restrictors may be made of sapphire, metal or plastic (other materials are also contemplated).

Flow of oxygen enriched air between the canisters is also controlled by use of a two-way valve 154 (labelled G). Valve 154 may be opened during the venting process and may be closed otherwise to prevent excessive oxygen loss out of the purging canister. Other durations are also contemplated. In an exemplary implementation, canister 302 is being vented and it is desirable to purge canister 302 by passing a portion of the oxygen enriched air being produced in canister 304 into canister 302. A portion of oxygen enriched air is passed into canister 302, from canister 304, through valve 154 and flow restrictors 153 and 155. The selection of appropriate flow restrictors 153 and 155, coupled with controlled opening of valve 154 allows a controlled amount of oxygen enriched air to be passed from canister 304 to purge canister 302. In an implementation, the controlled amount of oxygen enriched air is an amount sufficient to purge canister 302 and minimize the loss of oxygen enriched air to atmosphere through valve 122 of canister 302. While this implementation describes venting of canister 302, it should be understood that the same process can be used to vent canister 304 using valve 154 and flow restrictors 153 and 155.

Valve 154 works with flow restrictors 153 and 155 to optimize the gas flow balance between the two canisters. This may allow for better flow control for purging one of the canisters with oxygen enriched air from the other of the canisters. It may also provide better flow direction between the two canisters. In some implementations, the purge flow pathway may not have flow restrictors but instead the G-valve may have built-in resistance to flow, or the purge flow pathway itself may have a narrow radius to provide flow resistance.

In some implementations, the purge flow is stopped by de-actuating valve 154 at the same time as valve 152 is de-actuated, to complete the isolation of the two canisters when the pressures therein are equalised.

FIG. 2 is an illustration of one complete PSA cycle 2000 of a PSA process according to one implementation of the present technology. The diagram contains valve actuation waveforms 2010, 2020, 2030, and 2040 for the A-, B-, E-, and G-valves 122, 124, 152, and 154 respectively, that represent valve control signals generated by the controller 400. The diagram also contains pressure waveforms 2050 and 2060 indicative of the pressure in canisters 302 and 304 synchronised with the waveforms 2010, 2020, 2030, and 2040.

The PSA cycle 2000 illustrated in FIG. 2 contains eight sequential phases, each phase corresponding to a particular set of valve states (actuated or de-actuated) (e.g., respectively associated with high and low states of the signal of the waveform). The PSA cycle starts with a pressurisation phase in which canister 302 (labelled A) is being pressurised by the de-actuation of the A-valve 122. The pressure waveform 2050 indicates a steady rise in the pressure of canister 302. Meanwhile canister 304 (labelled B) is being vented by the actuation of the B-valve 124. The pressure waveform 2060 indicates a rapid depressurisation of canister 304. Thus, the pressurisation phase of canister 302 coincides with the desorb/vent phase of canister 304. The E- and G-valves 152 and 154 are de-actuated to prevent any interchange of gas between the canisters at their product ends.

The second phase begins when the G-valve 154 is actuated, allowing a portion of the oxygen enriched air leaving canister 302 to be purge desorbed nitrogen and other gases from canister 304. The pressure in canister 302 stabilises, and canister 302 continues to adsorb nitrogen and produce oxygen enriched air. Meanwhile the pressure in canister 304 rises a little, and then stabilises. This phase is referred to as the adsorption phase for canister 302, and the purge phase for canister 304.

The third phase is triggered by the actuation of the E-valve 152, which directly connects canister 302 to 304 at their product ends. This causes the pressure in canister 302 to fall rapidly while the pressure in canister 304 rises equally rapidly towards equilibrium with canister 302. The third phase is referred to as the equalisation (1) phase for canister 302. A short time later, the fourth phase commences when the B-valve 124 is de-actuated, ending the venting of canister 304 and connecting compression system 200 to canister 304 to assist with this equalisation of pressures from the feed end. The pressures in canisters 302 and 304 continue to fall and rise respectively. The fourth phase is referred to as the equalisation (2) phase for canister 302.

The fifth phase commences at the end of the equalisation (2) phase when the pressures in canisters 302 and 304 are approximately equal. The A-valve 122 is actuated to disconnect canister 302 from the compression system 200 and connect it to atmosphere to allow exhaust gas to vent. Simultaneously, the G-valve 154 and the E-valve 152 are de-actuated to prevent any interchange of gas between the canisters at their product ends. The B-valve 124 remains de-actuated. The pressure waveform 2050 indicates a rapid depressurisation of canister 302. The pressure waveform 2060 indicates a steady rise in the pressure of canister 304. The fifth phase therefore mirrors the first phase with the roles of canisters 302 and 304 reversed. Thus, the pressurisation phase of canister 304 coincides with the desorb/vent phase of canister 302.

The sixth phase begins when the G-valve 154 is actuated, allowing a portion of the oxygen enriched air leaving canister 304 to be purge desorbed nitrogen and other gases from canister 302. The pressure in canister 304 stabilises, and canister 304 continues to adsorb nitrogen and produce oxygen enriched air. Meanwhile the pressure in canister 302 rises a little, and then stabilises. The sixth phase is referred to as the purge phase for canister 302, and the adsorption phase for canister 304.

The seventh phase is triggered by the actuation of the E-valve 152, which directly connects canister 302 to 304 at their product ends. This causes the pressure in canister 302 to rise rapidly while the pressure in canister 304 falls equally rapidly towards equilibrium with canister 304. The seventh phase is referred to as the equalisation (1) phase for canister 304. A short time later, the eighth phase commences when the A-valve 122 is de-actuated, ending the venting of canister 302 and connecting compression system 200 to canister 302 to assist with this equalisation of pressures from the feed end. The pressures in canisters 302 and 304 continue to rise and fall respectively. The eighth phase is referred to as the equalisation (2) phase for canister 304. The PSA cycle 2000 is then complete and the PSA process continues with another PSA cycle.

The first to fourth phases make up a PSA half cyle while the fifth to eighth phase make up the other PSA half cycle.

Table 1 contains base phase durations, in milliseconds, for a PSA half cycle in each of six flow rate settings according to one implementation of the present technology.

TABLE 1
Base phase durations, in milliseconds, for a PSA
half cycle at each of six flow rate settings

| | Pressurisation | Adsorption | Equalisation (1) | Equalisation (2) |
|---|---|---|---|---|
| | (phase 1 and | (phase 2 and | (phase 3 and | (phase 4 and |
| Flow rate | corresponding | corresponding | corresponding | corresponding |
| setting | phase 5) | phase 6) | phase 7) | phase 8) |
| 1 | 2800 | 2800 | 100 | 500 |
| 2 | 2600 | 2600 | 100 | 500 |
| 3 | 2500 | 2500 | 100 | 500 |
| 4 | 2300 | 2300 | 100 | 500 |
| 5 | 2100 | 2100 | 100 | 500 |
| 6 | 2100 | 2100 | 100 | 500 |

In some implementations, the phase durations for a full PSA cycle are identical (and equal to the base phase durations) between the two PSA half cycles. However, in some implementations, there are differences in the phase durations between the two half cycles of a full PSA cycle. Table 2 contains static adjustments to the base durations of each phase (applied across all flow rate settings) in one implementation of the present technology.

TABLE 2
Static adjustments to phase durations of
each PSA cycle phase, in milliseconds.

| Phase | Static adjustment (ms) |
|---|---|
| 1 | 0 |
| 2 | 0 |
| 3 | 20 |
| 4 | 0 |
| 5 | 0 |
| 6 | 0 |
| 7 | 0 |
| 8 | 0 |

Static adjustments may be predetermined based on knowledge of fixed asymmetries between the pneumatic paths associated with canisters 302 and 304. Such asymmetries may arise because of impedance differences between the flow paths including each canister as a result of manufacturing tolerances. For example, the static adjustment to the duration of phase 3 (the equalisation (1) phase of canister 302) according to Table 2 is 20 ms. This means that phase 3 is statically 20 ms longer than the base duration of phases 3 and 7. Since the static adjustment to the duration of phase 7 is 0, this means that phase 3 is statically 20 ms longer than phase 7. Using the base phase duration values in Table 1, this means that the static durations of phase 3 and phase 7 are 120 ms and 100 ms respectively. Such a difference counteracts an asymmetry in the E-valve 152, which has higher impedance in the direction from canister 302 to 304 than it does in the direction from canister 304 to 302. The equalisation (1) phase for canister 302 therefore needs to be slightly longer to achieve the same volume of equalisation flow from canister 302 to canister 304 as from canister 304 to canister 302 during the equalisation (1) phase of canister 304.

As will be described below, the phase durations may also be adjusted dynamically to counteract dynamic imbalances between the pneumatic paths associated with canisters 302 and 304. Such adjustments may be made during patient use of the POC device such as where such imbalances develop over time of use.

The PSA cycle described above may be implemented by a PSA state machine.

Compression System

Referring to FIG. 1C, an implementation of an oxygen concentrator 100 is depicted. Oxygen concentrator 100 includes a compression system 200, a canister system 300, and a power supply 180 disposed within an outer housing 170. Inlets 101 are located in outer housing 170 to allow air from the environment to enter oxygen concentrator 100. Inlets 101 may allow air to flow into the compartment to assist with cooling of the components in the compartment. Power supply 180 provides a source of power for the oxygen concentrator 100. Compression system 200 draws air in through the inlet 105 and muffler 108. Muffler 108 may reduce noise of air being drawn in by the compression system and also may include a desiccant material to remove water from the incoming air. Oxygen concentrator 100 may further include fan 172 used to vent air and other gases from the oxygen concentrator via outlet 173.

In some implementations, compression system 200 includes one or more compressors. In another implementation, compression system 200 includes a single compressor, coupled to all of the canisters of canister system 300. Turning to FIGS. 1D and 1E, a compression system 200 is depicted that includes compressor 210 and motor 220. Motor 220 is coupled to compressor 210 and provides an operating force to the compressor to operate the compression mechanism. For example, motor 220 may be a motor providing a rotatable component that causes cyclical motion of a component of the compressor that compresses air. When compressor 210 is a piston type compressor, motor 220 provides an operating force which causes the piston of compressor 210 to be reciprocated. Reciprocation of the piston causes compressed air to be produced by compressor 210. The pressure of the compressed air is, in part, estimated by the speed that the compressor is operated at, (e.g., how fast the piston is reciprocated). Motor 220, therefore, may be a variable speed motor that is operable at various speeds to dynamically control the pressure of air produced by compressor 210.

In one implementation, compressor 210 includes a single head wobble type compressor having a piston. Other types of compressors may be used such as diaphragm compressors and other types of piston compressors. Motor 220 may be a DC or AC motor and provides the operating power to the compressing component of compressor 210. Motor 220, in an implementation, may be a brushless DC motor. Motor 220 may be a variable speed motor configured to operate the compressing component of compressor 210 at variable speeds. Motor 220 may be coupled to controller 400, which sends operating signals to the motor to control the operation of the motor. For example, controller 400 may send signals to motor 220 to: turn the motor on, turn motor the off, and set the operating speed of the motor. Thus, as illustrated in FIG. 1B, the compression system 200 may include a speed sensor 201. The speed sensor 201 may be a motor speed transducer used to determine a rotational speed of the motor 220 or a frequency of another reciprocating operation of the compression system 200. For example, a motor speed signal from the motor speed transducer (speed sensor 201) may be provided to the controller 400. The speed sensor 201 or motor speed transducer may, for example, be a Hall effect sensor. The controller 400 may operate the compression system 200 via the motor 220 based on the speed signal and/or any other sensor signal of the oxygen concentrator 100, such as a pressure sensor (e.g., accumulator pressure sensor 107). Thus, the controller 400 receives sensor signals, such as a speed signal from the speed sensor 201 and an accumulator pressure signal from the accumulator pressure sensor 107. With such signal(s), the controller 400 may implement one or more control loops (e.g., feedback control) for operation of the compression system 200 as described in more detail herein.

FIG. 3 is a schematic diagram of an example motor control circuit 3000 according to one implementation of the present technology in which the operating speed of the motor 220 is regulated to a speed set point while the motor 220 drives a load 290 including the compressor 210. Such speed control may be implemented by feedback control (closed-loop). The size of the load 290 is representative of the back pressure experienced by the compressor 210 while it generates the air stream. The back pressure in turn is related to the pressure within whichever of the canisters is being pressurised by the compressor 210.

In the motor control circuit 3000, a speed set point is provided to a motor controller 270 as a speed command 3010, e.g. by the POC controller 400. The setting of the speed set point is described in more detail below. The motor controller 270 may be implemented as an integrated circuit including, for example, one or more field programmable gate arrays (FPGAs), microcontrollers, etc. included on a circuit board disposed in the oxygen concentrator 100. Alternatively, the motor controller may be implemented as part of the controller 400, configured by program instructions stored in internal memory 420 or an external memory medium coupled to controller 400, and executed by one or more processors 410.

The motor controller 270 also takes as input a speed signal 3020 from the speed sensor 201. The motor controller processes the speed signal 3020 and the speed command 3010 and generates a motor control signal 3030. The motor control signal 3030 is thus generated with a characteristic parameter that permits control of the motor 220 so as to drive the load 290 at the speed set point given by the speed command 3010. As long as the speed set point is fixed, the characteristic parameter of the motor control signal 3030 is representative of the size of the load 290 at any time. Since power is load multiplied by speed, which is roughly constant, the characteristic parameter is representative of the power being developed by the motor 220 and may be referred to as the power parameter.

As mentioned above, the load 290 is representative of the pressure within whichever of the canisters 302 and 304 is connected to the compressor 210 via its inlet valve 122 or 124. The power parameter of the motor control signal 3030 is therefore representative of the pressure within the canister currently connected to the compressor 210.

In one implementation, the motor control signal 3030 is a bi-valued (high or low) waveform consisting of a train of pulses at a predetermined frequency that is independent of the motor speed. In one implementation, the pulse frequency is 20 kHz. The duty cycle of the pulse train (ratio or proportion of high time during one period to the duration of one period) ranges between 0% (no pulses at all) and 100% (one continuous pulse). Such a waveform is referred to as a pulse-width modulation (PWM) waveform. The duty cycle of the PWM waveform is the power parameter of the PWM waveform. In this implementation, the motor controller 270 generates a PWM waveform with a duty cycle such that the motor 220 is able to drive the load 290 at the speed set point given by the speed command 3010. As long as the speed set point is fixed, the duty cycle of the PWM waveform at any time is therefore representative of the size of the load 290 at that time. The duty cycle of the PWM waveform (the power parameter of the motor control signal 3030) is therefore representative of the pressure within the canister currently connected to the compressor 210 via its inlet valve.

In other implementations, the motor control signal 3030 is a continuously- or discretely-valued DC signal such as a voltage or current. In such implementations, the power parameter may be the value of the motor control signal 3030 itself.

Returning to FIG. 3, the motor control signal 3030 is passed to a motor driver circuit 280 that generates one or more motor drive signals 3040. The motor driver circuit 280 may be implemented as an integrated circuit including, for example, one or more field programmable gate arrays (FPGAs), microcontrollers, etc. included on a circuit board disposed in the oxygen concentrator 100. The motor control signal 3030 modulates the motor drive signals 3040 to adjust the amount of power supplied to the motor 220 and hence the speed at which the motor 220 drives the load 290. In one example implementation, illustrated in FIG. 3, the motor 220 is a three-phase motor so there are three differently-phased motor drive signals 3040, one for each winding.

### Heat Management

Compression system 200 inherently creates substantial heat. Heat is caused by the consumption of power by motor 220 and the conversion of power into mechanical motion. Compressor 210 generates heat due to the increased resistance to movement of the compressor components by the air being compressed. Heat is also inherently generated due to adiabatic compression of the air by compressor 210. Thus, the continual pressurization of air produces heat in the enclosure. Additionally, power supply 180 may produce heat as power is supplied to compression system 200. Furthermore, users of the oxygen concentrator may operate the device in unconditioned environments (e.g., outdoors) at potentially higher ambient temperatures than indoors, thus the incoming air will already be in a heated state.

Heat produced inside oxygen concentrator 100 can be problematic. Lithium ion batteries are generally employed as power supplies for oxygen concentrators due to their long life and light weight. Lithium ion battery packs, however, are dangerous at elevated temperatures and safety controls are employed in oxygen concentrator 100 to shutdown the system if dangerously high power supply temperatures are detected. Additionally, as the internal temperature of oxygen concentrator 100 increases, the amount of oxygen generated by the concentrator may decrease. This is due, in part, to the decreasing amount of oxygen in a given volume of air at higher temperatures. If the amount of produced oxygen drops below a predetermined amount, the oxygen concentrator 100 may automatically shut down.

Because of the compact nature of oxygen concentrators, dissipation of heat can be difficult. Solutions typically involve the use of one or more fans to create a flow of cooling air through the enclosure. Such solutions, however, require additional power from the power supply 180 and thus shorten the portable usage time of the oxygen concentrator. In an implementation, a passive cooling system may be used that takes advantage of the mechanical power produced by motor 220. Referring to FIGS. 1D and 1E, compression system 200 includes motor 220 having an external rotatable armature (or external rotatable armature) 230. Specifically, armature 230 of motor 220 (e.g. a DC motor) is wrapped around the stationary field that is driving the armature. Since motor 220 is a large contributor of heat to the overall system it is helpful to transfer heat off the motor and sweep it out of the enclosure. With the external high speed rotation, the relative velocity of the major component of the motor and the air in which it exists is very high. The surface area of the armature is larger if externally mounted than if it is internally mounted. Since the rate of heat exchange is proportional to the surface area and the square of the velocity, using a larger surface area armature mounted externally increases the ability of heat to be dissipated from motor 220. The gain in cooling efficiency by mounting the armature externally, allows the elimination of one or more cooling fans, thus reducing the weight and power consumption while maintaining the interior of the oxygen concentrator within the appropriate temperature range. Additionally, the rotation of the externally mounted armature creates movement of air proximate to the motor to create additional cooling.

Moreover, an external rotatable armature may help the efficiency of the motor, allowing less heat to be generated. A motor having an external armature operates similar to the way a flywheel works in an internal combustion engine. When the motor is driving the compressor, the resistance to rotation is low at low pressures. When the pressure of the compressed air is higher, the resistance to rotation of the motor is higher. As a result, the motor does not maintain consistent ideal rotational stability, but instead surges and slows down depending on the pressure demands of the compressor. This tendency of the motor to surge and then slow down is inefficient and therefore generates heat. Use of an external armature adds greater angular momentum to the motor which helps to compensate for the variable resistance experienced by the motor. Since the motor does not have to work as hard, the heat produced by the motor may be reduced.

In an implementation, cooling efficiency may be further increased by coupling an air transfer device 240 to external rotatable armature 230. In an implementation, air transfer device 240 is coupled to the external armature 230 such that rotation of the external armature 230 causes the air transfer device 240 to create an air flow that passes over at least a portion of the motor. In an implementation, air transfer device 240 includes one or more fan blades coupled to the external armature 230. In an implementation, a plurality of fan blades may be arranged in an annular ring such that the air transfer device 240 acts as an impeller that is rotated by movement of the external rotatable armature 230. As depicted in FIGS. 1D and 1E, air transfer device 240 may be mounted to an outer surface of the external armature 230, in alignment with the motor 220. The mounting of the air transfer device 240 to the armature 230 allows air flow to be directed toward the main portion of the external rotatable armature 230, providing a cooling effect during use. In an implementation, the air transfer device 240 directs air flow such that a majority of the external rotatable armature 230 is in the air flow path.

Further, referring to FIGS. 1D and 1E, air pressurized by compressor 210 exits compressor 210 at compressor outlet 212. A compressor outlet conduit 250 is coupled to compressor outlet 212 to transfer the compressed air to canister system 300. As noted previously, compression of air causes an increase in the temperature of the air. This increase in temperature can be detrimental to the efficiency of the oxygen concentrator. In order to reduce the temperature of the pressurized air, compressor outlet conduit 250 is placed in the air flow path produced by air transfer device 240. At least a portion of compressor outlet conduit 250 may be positioned proximate to motor 220. Thus, air flow, created by air transfer device 240, may contact both motor 220 and compressor outlet conduit 250. In one implementation, a majority of compressor outlet conduit 250 is positioned proximate to motor 220. In an implementation, the compressor outlet conduit 250 is coiled around motor 220, as depicted in FIG. 1E.

In an implementation, the compressor outlet conduit 250 is composed of a heat exchange metal. Heat exchange metals include, but are not limited to, aluminum, carbon steel, stainless steel, titanium, copper, copper-nickel alloys or other alloys formed from combinations of these metals. Thus, compressor outlet conduit 250 can act as a heat exchanger to remove heat that is inherently caused by compression of the air. By removing heat from the compressed air, the number of molecules in a given volume at a given pressure is increased. As a result, the amount of oxygen enriched air that can be generated by each canister during each PSA cycle may be increased.

The heat dissipation mechanisms described herein are either passive or make use of elements required for the oxygen concentrator 100. Thus, for example, dissipation of heat may be increased without using systems that require additional power. By not requiring additional power, the run-time of the battery packs may be increased and the size and weight of the oxygen concentrator may be minimized. Likewise, use of an additional box fan or cooling unit may be eliminated. Eliminating such additional features reduces the weight and power consumption of the oxygen concentrator.

As discussed above, adiabatic compression of air causes the air temperature to increase. During venting of a canister in canister system 300, the pressure of the exhaust gas being vented from the canisters decreases. The adiabatic decompression of the gas leaving the canister causes the temperature of the exhaust gas to drop as it is vented. In an implementation, the cooled exhaust gas 327 vented from canister system 300 is directed toward power supply 180 and toward compression system 200. In an implementation, base 315 of canister system 300 receives the exhaust gas from the canisters. The exhaust gas 327 is directed through base 315 toward outlet 325 of the base 315 and toward power supply 180. The exhaust gas, as noted, is cooled due to decompression of the gases and therefore passively provide cooling to the power supply 180. When the compression system 200 is operated, the air transfer device 240 will gather the cooled exhaust gas 327 and direct the exhaust gas 327 toward the motor 220 of compression system 200. Fan 172 may also assist in directing the exhaust gas 327 across compression system 200 and out of the housing 170. In this manner, additional cooling may be obtained without requiring any further power from the battery.

Canister System

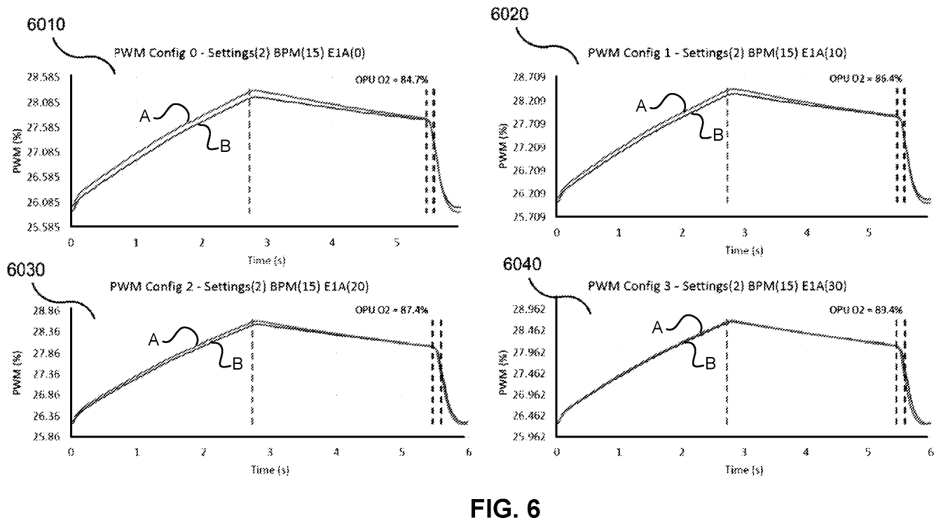

Oxygen concentrator 100 may include at least two canisters, each canister including a gas separation adsorbent. The canisters of oxygen concentrator 100 may be formed from a molded housing. In an implementation, canister system 300 includes two housing components 310 and 510, as depicted in FIG. 11. In various implementations, the housing components 310 and 510 of the oxygen concentrator 100 may form a two-part molded plastic frame that defines two canisters 302 and 304 and accumulator 106. The housing components 310 and 510 may be formed separately and then coupled together. In some implementations, housing components 310 and 510 may be injection molded or compression molded. Housing components 310 and 510 may be made from a thermoplastic polymer such as polycarbonate, methylene carbide, polystyrene, acrylonitrile butadiene styrene (ABS), polypropylene, polyethylene, or polyvinyl chloride. In another implementation, housing components 310 and 510 may be made of a thermoset plastic or metal (such as stainless steel or a lightweight aluminum alloy). Lightweight materials may be used to reduce the weight of the oxygen concentrator 100. In some implementations, the two housing components 310 and 510 may be fastened together using screws or bolts. Alternatively, housing components 310 and 510 may be laser or solvent welded together.

As shown, valve seats 322, 324, 332, and 334 and conduits 330 and 346 may be integrated into the housing component 310 to reduce the number of sealed connections needed throughout the air flow of the oxygen concentrator 100.

Air pathways/tubing between different sections in housing components 310 and 510 may take the form of molded conduits. Conduits in the form of molded channels for air pathways may occupy multiple planes in housing components 310 and 510. For example, the molded air conduits may be formed at different depths and at different positions in housing components 310 and 510. In some implementations, a majority or substantially all of the conduits may be integrated into the housing components 310 and 510 to reduce potential leak points.

In some implementations, prior to coupling housing components 310 and 510 together, 0-rings may be placed between various points of housing components 310 and 510 to ensure that the housing components are properly sealed. In some implementations, components may be integrated and/or coupled separately to housing components 310 and 510. For example, tubing, flow restrictors (e.g., press fit flow restrictors), oxygen sensors, gas separation adsorbents, check valves, plugs, processors, power supplies, etc. may be coupled to housing components 310 and 510 before and/or after the housing components are coupled together.

In some implementations, apertures 337 leading to the exterior of housing components 310 and 510 may be used to insert devices such as flow restrictors. Apertures may also be used for increased moldability. One or more of the apertures may be plugged after molding (e.g., with a plastic plug). In some implementations, flow restrictors may be inserted into passages prior to inserting plugs to seal the passages. Press fit flow restrictors may have diameters that may allow a friction fit between the press fit flow restrictors and their respective apertures. In some implementations, an adhesive may be added to the exterior of the press fit flow restrictors to hold the press fit flow restrictors in place once inserted. In some implementations, the plugs may have a friction fit with their respective tubes (or may have an adhesive applied to their outer surface). The press fit flow restrictors and/or other components may be inserted and pressed into their respective apertures using a narrow tip tool or rod (e.g., with a diameter less than the diameter of the respective aperture). In some implementations, the press fit flow restrictors may be inserted into their respective tubes until they abut a feature in the tube to halt their insertion. For example, the feature may include a reduction in radius. Other features are also contemplated (e.g., a bump in the side of the tubing, threads, etc.). In some implementations, press fit flow restrictors may be molded into the housing components (e.g., as narrow tube segments).

In some implementations, spring baffle 139 may be placed into respective canister receiving portions of housing components 310 and 510 with the spring side of the baffle 139 facing the exit of the canister. Spring baffle 139 may apply force to gas separation adsorbent in the canister while also assisting in preventing gas separation adsorbent from entering the exit apertures. Use of a spring baffle 139 may keep the gas separation adsorbent compact while also allowing for expansion (e.g., thermal expansion). Keeping the gas separation adsorbent compact may prevent the gas separation adsorbent from breaking during movement of the oxygen concentrator 100.

In some implementations, filter 129 may be placed into respective canister receiving portions of housing components 310 and 510 facing the inlet of the respective canisters. The filter 129 removes particles from the air stream entering the canisters.

In some implementations, pressurized air from the compression system 200 may enter air inlet 306. Air inlet 306 is coupled to inlet conduit 330. Air enters housing component 310 through inlet 306 and travels through inlet conduit 330, and then to valve seats 322 and 324. FIG. 1J and FIG. 1K depict an end view of housing component 310. FIG. 1J depicts an end view of housing component 310 prior to fitting valves to housing component 310. FIG. 1K depicts an end view of housing component 310 with the valves fitted to the housing component 310. Valve seats 322 and 324 are configured to receive inlet valves 122 and 124 respectively. Inlet valve 122 is coupled to canister 302 and inlet valve 124 is coupled to canister 304.

In an implementation, pressurized air is sent into one of canisters 302 or 304 while the other canister is being vented. Valve seat 322 includes an opening 323 that passes through housing component 310 into canister 302. Similarly valve seat 324 includes an opening 375 that passes through housing component 310 into canister 304. Air from inlet conduit 330 passes through openings 323 or 375 if the respective valves 122 and 124 are de-actuated, and enters the respective canisters 302 and 304.

Check valves 142 and 144 (See FIG. 11) are coupled to canisters 302 and 304, respectively. Check valves 142 and 144 are one-way valves that are passively operated by the pressure differentials that occur as the canisters are pressurized and vented. Oxygen enriched air produced in canisters 302 and 304 passes from the canisters into openings 542 and 544 of housing component 510. A passage (not shown) links openings 542 and 544 to conduits 342 and 344, respectively. Oxygen enriched air produced in canister 302 passes from the canister though opening 542 and into conduit 342 when the pressure in the canister is sufficient to open check valve 142. When check valve 142 is open, oxygen enriched air flows through conduit 342 toward the end of housing component 310. Similarly, oxygen enriched air produced in canister 304 passes from the canister through opening 544 and into conduit 344 when the pressure in the canister is sufficient to open check valve 144. When check valve 144 is open, oxygen enriched air flows through conduit 344 toward the end of housing component 310.

Oxygen enriched air from either canister travels through conduit 342 or 344 and enters conduit 346 formed in housing component 310. Conduit 346 includes openings that couple the conduit to conduit 342, conduit 344 and accumulator 106. Thus, oxygen enriched air, produced in canister 302 or 304, travels to conduit 346 and passes into accumulator 106. As illustrated in FIG. 1B, gas pressure within the accumulator 106 may be measured by a sensor, such as with an accumulator pressure sensor 107. (See also FIG. 1F.) Thus, the accumulator pressure sensor 107 generates a signal representing the pressure of the accumulated oxygen enriched air. An example of a suitable pressure transducer is a sensor from the HONEYWELL ASDX series. An alternative suitable pressure transducer is a sensor from the NPA Series from GENERAL ELECTRIC. In some versions, the pressure sensor 107 may alternatively measure pressure of the gas outside of the accumulator 106, such as in an output path between the accumulator 106 and a valve (e.g., supply valve 160) that controls the release of the oxygen enriched air for delivery to a user in a bolus.

Canister 302 is vented by actuating inlet valve 122, releasing the exhaust gas from canister 302 into the volume defined by the end of housing component 310. Foam material may cover the end of housing component 310 to reduce the sound made by release of gases from the canisters. Similarly, canister 304 is vented by actuating inlet valve 124, releasing the exhaust gas from canister 304 into the volume defined by the end of housing component 310.

Two conduits are formed in housing component 510 for use in transferring oxygen enriched air between canisters. As shown in FIG. 1L, conduit 530 couples canister 302 to canister 304. Conduit 530 is coupled to valve seat 554 which receives valve 154, as shown in FIG. 1M. Flow restrictors 153 and 155 (not shown) are disposed in conduit 530, between canister 302 and canister 304, to restrict flow of oxygen enriched air during purging. The valve 154 works with flow restrictors 153 and 155 to optimize the purge flow balance between the two canisters. Conduit 532 also couples canister 302 to 304. Conduit 532 is coupled to valve seat 552 which receives valve 152, as shown in FIG. 1M.

Oxygen enriched air in accumulator 106 passes through supply valve 160 as described below. An opening (not shown) in housing component 510 couples accumulator 106 to supply valve 160.

### Outlet System

An outlet system, coupled to one or more of the canisters, includes one or more conduits for providing oxygen enriched air to a user. In an implementation, oxygen enriched air produced in either of canisters 302 and 304 is collected in accumulator 106 through check valves 142 and 144, respectively, as depicted schematically in FIG. 1B. The oxygen enriched air leaving the canisters may be collected in an oxygen accumulator 106 prior to being provided to a user. In some implementations, a conduit may be coupled to the accumulator 106 to provide the oxygen enriched air to the user. Oxygen enriched air may be provided to the user through an airway delivery device that transfers the oxygen enriched air to the user's mouth and/or nose. In an implementation, a delivery device may include a tube that directs the oxygen toward a user's nose and/or mouth that may not be directly coupled to the user's nose.

Turning to FIG. 1F, a schematic diagram of an implementation of an outlet system 150 for an oxygen concentrator is shown. A supply valve 160 may be coupled a conduit to control the release of the oxygen enriched air from accumulator 106 to the user. Supply valve 160 is actuated by controller 400 to control the delivery of oxygen enriched air to a user. In an implementation, supply valve 160 is an electromagnetically actuated plunger valve. Actuation of supply valve 160 is not timed or synchronized to the pressure swing adsorption process. Instead, actuation is synchronized to the user's breathing as described below. In some implementations, supply valve 160 may have continuously-valued actuation to establish a clinically effective amplitude profile for providing oxygen enriched air.

Oxygen enriched air in accumulator 106 passes through supply valve 160 into oxygen sensor 165 as depicted in FIG. 1F. In an implementation, oxygen sensor 165 may include one or more devices configured to estimate an oxygen concentration (purity) of gas passing through the oxygen sensor 165. Oxygen enriched air is released from accumulator 106 by supply valve 160, and then is bled through a small orifice flow restrictor 175 to oxygen sensor 165 and then to particulate filter 187. Flow restrictor 175 may be a 0.025 D flow restrictor. Other flow restrictor types and sizes may be used. In some implementations, the diameter of the air pathway in the housing may be restricted to create restricted gas flow. Particulate filter 187 may be used to filter bacteria, dust, granule particles, etc., prior to delivery of the oxygen enriched air to the user. The oxygen enriched air passes through filter 187 to connector 190 which sends the oxygen enriched air to the user via delivery conduit 192 and to pressure sensor 194.

The fluid dynamics of the outlet pathway, coupled with the programmed actuations of supply valve 160, may result in a bolus of oxygen being provided at the correct time and with an amplitude profile that assures rapid delivery into the user's lungs without excessive waste.

Oxygen sensor 165 is a device configured to measure oxygen concentration in a gas. Examples of oxygen sensors include, but are not limited to, ultrasonic oxygen sensors, electrical oxygen sensors, chemical oxygen sensors, and optical oxygen sensors. In one implementation, oxygen sensor 165 is a chemical oxygen sensor

Particulate filter 187 removes bacteria, dust, granule particles, etc prior to providing the oxygen enriched air to the user. The filtered oxygen enriched air passes to connector 190. Connector 190 may be a “Y” connector coupling the outlet of filter 187 to pressure sensor 194 and delivery conduit 192. Pressure sensor 194 may be used to monitor the pressure of the gas passing through delivery conduit 192 to the user. In some implementations, pressure sensor 194 is configured to generate a signal that is proportional to the amount of positive or negative pressure applied to a sensing surface. Changes in pressure, sensed by pressure sensor 194, may be used to determine a breathing rate of a user, as well as the onset of inhalation (also referred to as the trigger instant) as described below. Controller 400 may control actuation of supply valve 160 based on the breathing rate and/or onset of inhalation of the user. In an implementation, controller 400 may control actuation of supply valve 160 based on information provided by the pressure sensor 194.

Oxygen enriched air may be provided to a user through delivery conduit 192. In an implementation, delivery conduit 192 may be a silicone tube. Delivery conduit 192 may be coupled to a user using an airway delivery device 196, as depicted in FIGS. 1G and 1H. An airway delivery device 196 may be any device capable of providing the oxygen enriched air to nasal cavities or oral cavities. Examples of airway delivery devices include, but are not limited to: nasal masks, nasal pillows, nasal prongs, nasal cannulas, and mouthpieces. A nasal cannula airway delivery device 196 is depicted in FIG. 1G. Nasal cannula airway delivery device 196 is positioned proximate to a user's airway (e.g., proximate to the user's mouth and or nose) to allow delivery of the oxygen enriched air to the user while allowing the user to breathe air from the surroundings.

In an alternate implementation, a mouthpiece may be used to provide oxygen enriched air to the user. As shown in FIG. 1H, a mouthpiece 198 may be coupled to oxygen concentrator 100. Mouthpiece 198 may be the only device used to provide oxygen enriched air to the user, or a mouthpiece may be used in combination with a nasal delivery device (e.g., a nasal cannula). As depicted in FIG. 1H, oxygen enriched air may be provided to a user through both nasal cannula airway delivery device 196 and mouthpiece 198.

Mouthpiece 198 is removably positionable in a user's mouth. In one implementation, mouthpiece 198 is removably couplable to one or more teeth in a user's mouth. During use, oxygen enriched air is directed into the user's mouth via the mouthpiece. Mouthpiece 198 may be a night guard mouthpiece which is molded to conform to the user's teeth. Alternatively, mouthpiece may be a mandibular repositioning device. In an implementation, at least a majority of the mouthpiece is positioned in a user's mouth during use.

During use, oxygen enriched air may be directed to mouthpiece 198 when a change in pressure is detected proximate to the mouthpiece. In one implementation, mouthpiece 198 may be coupled to a pressure sensor 194. When a user inhales air through the user's mouth, pressure sensor 194 may detect a drop in pressure proximate to the mouthpiece. Controller 400 of oxygen concentrator 100 may control release of a bolus of oxygen enriched air to the user at the onset of inhalation.

During typical breathing of an individual, inhalation may occur through the nose, through the mouth or through both the nose and the mouth. Furthermore, breathing may change from one passageway to another depending on a variety of factors. For example, during more active activities, a user may switch from breathing through their nose to breathing through their mouth, or breathing through their mouth and nose. A system that relies on a single mode of delivery (either nasal or oral), may not function properly if breathing through the monitored pathway is stopped. For example, if a nasal cannula is used to provide oxygen enriched air to the user, an inhalation sensor (e.g., a pressure sensor or flow rate sensor) is coupled to the nasal cannula to determine the onset of inhalation. If the user stops breathing through their nose, and switches to breathing through their mouth, the oxygen concentrator 100 may not know when to provide the oxygen enriched air since there is no feedback from the nasal cannula. Under such circumstances, oxygen concentrator 100 may increase the flow rate and/or increase the frequency of providing oxygen enriched air until the inhalation sensor detects an inhalation by the user. If the user switches between breathing modes often, the default mode of providing oxygen enriched air may cause the oxygen concentrator 100 to work harder, limiting the portable usage time of the system.

In an implementation, mouthpiece 198 is used in combination with nasal cannula airway delivery device 196 to provide oxygen enriched air to a user, as depicted in FIG. 1H. Both mouthpiece 198 and nasal cannula airway delivery device 196 are coupled to an inhalation sensor. In one implementation, mouthpiece 198 and nasal cannula airway delivery device 196 are coupled to the same inhalation sensor. In an alternate implementation, mouthpiece 198 and nasal cannula airway delivery device 196 are coupled to different inhalation sensors. In either implementation, the inhalation sensor(s) may detect the onset of inhalation from either the mouth or the nose. Oxygen concentrator 100 may be configured to provide oxygen enriched air to the delivery device (i.e. mouthpiece 198 or nasal cannula airway delivery device 196) proximate to which the onset of inhalation was detected. Alternatively, oxygen enriched air may be provided to both mouthpiece 198 and nasal cannula airway delivery device 196 if onset of inhalation is detected proximate either delivery device. The use of a dual delivery system, such as depicted in FIG. 1H may be particularly useful for users when they are sleeping and may switch between nose breathing and mouth breathing without conscious effort.

### Controller System

Operation of oxygen concentrator 100 may be performed automatically using an internal controller 400 coupled to various components of the oxygen concentrator 100, as described herein. The controller 400 may be implemented by one or more hardware components (e.g., hardware controller(s)) and may be implemented with one or more programming logic or software controllers that are programming logic modules of a hardware controller. Thus, controller 400 may include one or more processors 410 and internal memory 420, as depicted in FIG. 1B. Methods used to operate and monitor oxygen concentrator 100 may be implemented by program instructions stored in internal memory 420 or an external memory medium coupled to controller 400, and executed by one or more processors 410. A memory medium may include any of various types of memory devices or storage devices. The term “memory medium” is intended to include an installation medium, e.g., a Compact Disc Read Only Memory (CD-ROM), floppy disks, or tape device; a computer system memory or random access memory such as Dynamic Random Access Memory (DRAM), Double Data Rate Random Access Memory (DDR RAM), Static Random Access Memory (SRAM), Extended Data Out Random Access Memory (EDO RAM), Random Access Memory (RAM), etc.; or a non-volatile memory such as a magnetic medium, e.g., a hard drive, or optical storage. The memory medium may comprise other types of memory as well, or combinations thereof. In addition, the memory medium may be located proximate to the controller 400 by which the programs or control logic are executed, or may be located in an external computing device that connects to the controller 400 over a network, as described below. In the latter instance, the external computing device may provide program instructions to the controller 400 for execution. The term “memory medium” may include two or more memory media that may reside in different locations, e.g., in different computing devices that are connected over a network.

In some implementations, controller 400 includes processor 410 that includes, for example, one or more field programmable gate arrays (FPGAs), microcontrollers, etc. included on a circuit board disposed in oxygen concentrator 100. Processor 410 is configured to execute programming instructions (e.g., control logic) stored in memory 420. In some implementations, programming instructions may be built into processor 410 such that a memory external to the processor 410 may not be separately accessed (i.e., the memory 420 may be internal to the processor 410).

Processor 410 may be coupled to various components of oxygen concentrator 100, including, but not limited to compression system 200, one or more of the valves used to control fluid flow through the system (e.g., valves 122, 124, 152, 154, 160), oxygen sensor 165, pressure sensor 194, temperature sensors (not shown), fan 172, and any other component that may be electrically controlled. In some implementations, a separate processor (and/or memory) may be coupled to one or more of the components.

Controller 400 may be configured (e.g. programmed by program instructions) to operate oxygen concentrator 100 and is further configured to monitor the oxygen concentrator 100 such as for malfunction states or other process information. For example, in one implementation, controller 400 is programmed to trigger an alarm if the system is operating and no breathing is detected by the user for a predetermined amount of time. For example, if controller 400 does not detect a breath for a period of 75 seconds, an alarm LED may be lit and/or an audible alarm may be sounded. If the user has truly stopped breathing, for example, during a sleep apnea episode, the alarm may be sufficient to awaken the user, causing the user to resume breathing. The action of breathing may be sufficient for controller 400 to reset this alarm function. Alternatively, if the system is accidentally left on when delivery conduit 192 is removed from the user, the alarm may serve as a reminder for the user to turn oxygen concentrator 100 off.

Controller 400 is further coupled to oxygen sensor 165, and may be programmed for continuous or periodic monitoring of the oxygen concentration of the oxygen enriched air passing through oxygen sensor 165. A minimum oxygen concentration threshold may be programmed into controller 400, such that the controller lights an LED visual alarm and/or an audible alarm to warn the user of the low concentration of oxygen.

Controller 400 is also coupled to internal power supply 180 and may be configured to monitor the level of charge of the internal power supply. A minimum voltage and/or current threshold may be programmed into controller 400, such that the controller lights an LED visual alarm and/or an audible alarm to warn the user of low power condition. The alarms may be activated intermittently and at an increasing frequency as the battery approaches zero usable charge.

Further functions that may be implemented with or by the controller 400 are described in detail in other sections of this disclosure. For example, the controller of the POC may implement compressor control to regulate pressure in the system. Thus, the POC may be equipped with a pressure sensor such as the pressure sensor 107 in the accumulator 106 downstream of the canisters 302 and 304. The controller 400 in the POC can control adjusting of the speed of the compressor 210 using signals from the pressure sensor as well as a motor speed sensor such as in one or more modes. In this regard, the controller may implement dual control modes, designated a coarse pressure regulation mode and a fine pressure regulation mode. The coarse pressure regulation mode may be implemented for changing between the different flow rate settings (or “flow settings”) of the POC and for starting/initial activation. The fine pressure regulation mode may then take over upon completion of each operation of the coarse pressure regulation mode.

In the coarse pressure regulation mode, the motor speed is set/controlled to ramp up or down depending the prior state of operations. During the ramping, the controller uses the measurements from the pressure sensor to generate an estimated pressure upstream of the sensor, in the canisters. In some implementations, the estimated pressure is used in a test to terminate the ramp, e.g. when the estimated pressure reaches a predetermined target pressure value, created at manufacturing time, that is associated with the selected flow rate setting of the POC. Table 3 contains flow rates and target pressure values associated with each of six flow rate settings according to one implementation of the present technology.

TABLE 3
Flow rates and target pressure values at each flow rate setting

| Flow rate setting | Flow rate (LPM) | Target pressure (kPa) |
|---|---|---|
| 1 | 0.2 | 45 |
| 2 | 0.4 | 60 |
| 3 | 0.6 | 75 |
| 4 | 0.8 | 95 |
| 5 | 1.0 | 115 |
| 6 | 1.1 | 125 |

The pressure estimate may be calculated by performing a regression (e.g., linear) using data from the pressure sensor whereby the controller determines regression parameters (e.g., slope and intercept parameters of a line) from the sensor signal samples. The pressure estimate is calculated with the regression parameters and a known system response delay.

In the fine pressure regulation mode, the motor speed is controlled to regulate the pressure of the system to the target pressure value using the signal from the pressure sensor. Upon completion of the coarse pressure regulation mode, the motor speed ramping is stopped and a base motor speed is set equal to the current motor speed. Any further changes to the motor speed are implemented by a fine pressure controller such as a PID (proportional, integral, derivative) controller. During the fine pressure regulation mode, the target pressure is compared with a qualified pressure estimate to generate a first error signal that is applied to the fine pressure controller to produce a speed adjustment. By adding the speed adjustment to the base motor speed, a speed set point for the motor may be obtained. The speed set point is then passed to the motor control circuit 3000 described above with reference to FIG. 3 which will cause the motor to operate at the speed set point and thereby achieve the target system pressure.

The qualified pressure estimate for the fine pressure controller may be computed using regression. In this regard, samples from the pressure signal may be applied to a best fit algorithm (e.g., linear regression) to determine regression parameters (e.g., slope and intercept of a line) of the data from the pressure signal during an adsorption phase of the PSA cycle. If the slope is positive, these parameters (slope and intercept rather than pressure samples from the pressure sensor) may then be applied with the particular time of the given adsorption phase of the PSA cycle to determine a peak value of the regression line from the linear regression. If the slope is negative, the intercept parameter may be taken as the peak value. The peak values from the regression information may be then applied to a running average buffer that maintains an average of the most recent peak values (e.g., six or more). The average peak value may then serve as the qualified pressure estimate for the fine pressure controller. Versions of such processes are discussed in more detail in U.S. Provisional Patent Application No. 62/904,858, filed on 24 Sep. 2019, the entire disclosure of which is incorporated herein by reference.

Additionally, the controller of the POC may be configured to implement supply valve control to regulate bolus size (volume) in the system, which may optionally be implemented without use of a flow rate sensor of the POC. For example, the POC may be equipped with a pressure sensor, such as the pressure sensor 107 in the accumulator 106 downstream of the canisters, and regulate bolus size, generated by the POC, as a function of pressure. Such regulation of bolus size may be a function of accumulator pressure.

Still further, as previously mentioned, the POC may implement dynamic imbalance reduction. Such dynamic imbalance reduction functionality may be implemented with or by the controller 400. Such functionality, which may include control logic therefor, is described in detail in other sections of this disclosure. For example, the controller 400 may be implemented as one or more controllers to regulate the speed of the compressor to a speed set point while generating the pressurised air stream by generating a motor control signal having a power parameter. The controller 400 may be implemented to selectively operate the one or more valves in a cyclic pattern so as to produce oxygen enriched air in the accumulator. A cycle of the cyclic pattern may have a plurality of phases, and each of the plurality of phases may have a duration that may be implemented by a duration setpoint. The controller 400 may generate dynamic adjustment(s) to one or more of the durations (e.g., the duration setpoint) based on evaluation of the power parameter. Such a dynamic adjustment may reduce dynamic imbalance of pneumatic characteristics, such as pressure, between the pneumatic paths associated with the canisters.

### Control Panel

Control panel 600 serves as an interface between a user and controller 400 to allow the user to initiate predetermined operation modes of the oxygen concentrator 100 and to monitor the status of the system. FIG. 1N depicts an implementation of control panel 600. Charging input port 605, for charging the internal power supply 180, may be disposed in control panel 600.

In some implementations, control panel 600 may include buttons to activate various operation modes for the oxygen concentrator 100. For example, control panel may include power button 610, flow rate setting buttons 620 to 626, active mode button 630, sleep mode button 635, altitude button 640, and a battery check button 650. In some implementations, one or more of the buttons may have a respective LED that may illuminate when the respective button is pressed, and may power off when the respective button is pressed again. Power button 610 may power the system on or off. If the power button 610 is activated to turn the system off, controller 400 may initiate a shutdown sequence to place the system in a shutdown state (e.g., a state in which both canisters are pressurized).

Flow rate setting buttons 620, 622, 624, and 626 allow a flow rate of oxygen enriched air to be selected (e.g., 0.2 LPM by button 620, 0.4 LPM by button 622, 0.6 LPM by button 624, and 0.8 LPM by button 626). In other implementations, the number of flow rate settings may be increased or decreased. After a flow rate setting is selected, oxygen concentrator 100 will then control operations to achieve production of the oxygen enriched air according to the selected flow rate setting.

Altitude button 640 may be activated when a user is going to be in a location at a higher elevation than the oxygen concentrator 100 is regularly used by the user.

Battery check button 650 initiates a battery check routine in the oxygen concentrator 100 which results in a relative battery power remaining LED 655 being illuminated on control panel 600.

A user may have a low breathing rate or depth if relatively inactive (e.g., asleep, sitting, etc.) as estimated by comparing the detected breathing rate or depth to a threshold. The user may have a high breathing rate or depth if relatively active (e.g., walking, exercising, etc.). An active/sleep mode may be estimated automatically from breathing rate or depth, and/or the user may manually indicate active mode or sleep mode by pressing button 630 for active mode or button 635 for sleep mode. In some implementations, the POC 100 defaults to active mode.

### Methods of Operating the POC

The methods of operating and monitoring the POC 100 described below may be executed by the one or more processors, such as the one or more processors 410 of the controller 400, configured by program instructions, such as including, as previously described, the one or more functions and/or associated data corresponding thereto, stored in a memory such as the memory 420 of the POC 100.

The main use of an oxygen concentrator 100 is to provide supplemental oxygen to a user. One or more flow rate settings may be selected on a control panel 600 of the oxygen concentrator 100, which then will control operations to achieve production of the oxygen enriched air according to the selected flow rate setting. In some versions, a plurality of flow rate settings may be implemented (e.g., five flow rate settings). As described in more detail herein, the controller 400 may implement a POD (pulsed oxygen delivery) or demand mode of operation. Controller 400 may regulate the size of the one or more released pulses or boluses to achieve delivery of the oxygen enriched air according to the selected flow rate setting.

In order to maximise the effect of the delivered oxygen enriched air, controller 400 may be programmed to synchronise the release of each bolus of the oxygen enriched air with the user's inhalations. Releasing a bolus of oxygen enriched air to the user as the user inhales may reduce wastage of oxygen by not releasing oxygen, for example, when the user is exhaling. The flow rate settings on the control panel 600 may correspond to minute volumes (bolus volume multiplied by breathing rate per minute) of delivered oxygen, e.g. as set out in Table 3: 0.2 LPM, 0.4 LPM, 0.6 LPM, 0.8 LPM, 1 LPM, 1.1 LPM.

Oxygen enriched air produced by oxygen concentrator 100 is stored in an oxygen accumulator 106 and, in a POD mode of operation, released to the user as the user inhales. The amount of oxygen enriched air provided by oxygen concentrator 100 is controlled, in part, by supply valve 160. In an implementation, supply valve 160 is opened for a sufficient amount of time to provide the appropriate amount of oxygen enriched air, as estimated by controller 400, to the user. In order to minimize the wastage of oxygen, the oxygen enriched air may be released as a bolus soon after the onset of a user's inhalation is detected. For example, the bolus of oxygen enriched air may be released in the first few milliseconds of a user's inhalation.

In an implementation, an inhalation sensor such as a pressure sensor 194 may be used to detect the onset of inhalation by the user (a process referred to as “triggering”). For example, the onset of the user's inhalation may be detected by using pressure sensor 194. In use, delivery conduit 192 for providing oxygen enriched air is coupled to the user's nose and/or mouth through the nasal airway delivery device 196 and/or mouthpiece 198. The pressure in delivery conduit 192 is therefore representative of the user's airway pressure and hence indicative of user respiration. At the onset of inhalation, the user begins to draw air into their body through the nose and/or mouth. As the air is drawn in, a negative pressure is generated at the end of delivery conduit 192, due, in part, to the venturi action of the air being drawn across the end of delivery conduit 192. Controller 400 analyses the pressure signal from the pressure sensor 194 to detect a drop in pressure indicating the onset of inhalation. Upon detection of the onset of inhalation, supply valve 160 is opened to release a bolus of oxygen enriched air from the accumulator 106.

A positive change or rise in the pressure in delivery conduit 192 indicates an exhalation by the user. Controller 400 may analyze the pressure signal from pressure sensor 194 to detect a rise in pressure indicating the onset of exhalation. In one implementation, when a positive pressure change is sensed, supply valve 160 is closed until the next onset of inhalation is detected. Alternatively, supply valve 160 may be closed after a predetermined interval known as the bolus duration.

By measuring the intervals between adjacent onsets of inhalation, the user's breathing rate may be estimated. By measuring the intervals between onsets of inhalation and the subsequent onsets of exhalation, the user's inspiratory time may be estimated.

In other implementations, the pressure sensor 194 may be located in a sensing conduit that is in pneumatic communication with the user's airway, but separate from the delivery conduit 192. In such implementations the pressure signal from the pressure sensor 194 is therefore also representative of the user's airway pressure.

In some implementations, the sensitivity of the pressure sensor 194 may be affected by the physical distance of the pressure sensor 194 from the user, especially if the pressure sensor 194 is located in oxygen concentrator 100 and the pressure difference is detected through delivery conduit 192 coupling the oxygen concentrator 100 to the user. In some implementations, the pressure sensor 194 may be placed in the airway delivery device 196 used to provide the oxygen enriched air to the user. A signal from the pressure sensor 194 may be provided to controller 400 in the oxygen concentrator 100 electronically via a wire or through telemetry such as through Bluetooth™ or other wireless technology.

The sensitivity of the triggering process is governed by a trigger threshold with which the signal from the pressure sensor 194 is compared to determine whether a significant drop in pressure indicating onset of inhalation has taken place. Adjusting the trigger threshold alters the sensitivity of the triggering process. In some implementations, the trigger threshold is set to give the triggering process a higher sensitivity when the POC 100 is in sleep mode (e.g. as estimated automatically or as requested by the user via the sleep mode button 635) compared to when the POC 100 is in active mode (e.g. as estimated automatically or as requested by the user via the active mode button 630).

In some implementations, if the POC 100 is in active mode and an onset of inhalation has not been detected for a predetermined interval, e.g. 8 seconds, the POC 100 changes to sleep mode, which increases the trigger sensitivity as described above. Then, if onset of inhalation is not detected for a further predetermined interval (e.g. 8 seconds), the POC 100 enters “auto-pulse” mode. In auto-pulse mode, the controller 400 controls actuation of the supply valve 160 so as to deliver boluses at regular, predetermined intervals, e.g. 4 seconds. The POC 100 exits auto-pulse mode once onset of inhalation is detected by the triggering process or the POC 100 is powered off.

In some implementations, if the user's current activity level, such as that estimated using the detected user's breathing rate, exceeds a predetermined threshold, controller 400 may implement an alarm (e.g., visual and/or audio) to warn the user that the current breathing rate is exceeding the delivery capacity of the oxygen concentrator 100. For example, the threshold may be set at 40 breaths per minute (BPM).

### Dynamic Phase Duration Adjustment for Imbalance Control

As mentioned above, the durations of each phase of the PSA cycle may be adjusted dynamically to counteract dynamic imbalances of pneumatic characteristics between the pneumatic paths associated with canisters 302 and 304. A principal imbalance of interest may be the imbalance between the pressures in the canisters over the PSA cycle, since this can be an important parameter in determining the production of oxygen enriched air by each canister. Dynamic imbalances may result from transient variations in leak from various points in the pneumatic paths. Another source of dynamic imbalance is a difference of impedance of the E-valve 152 between the flow direction from canister 302 to canister 304 and the flow direction from canister 304 to canister 302. Also, there may be periods of bias towards one canister as a result of the delivery of boluses in POD mode when the user's onset of inspiration happens to repeatedly coincide with the phases when that canister is principally responsible for the production of oxygen enriched air.

In one implementation, the pressure in each canister over the PSA cycle may be directly sensed, sampled, and converted to a difference value representative of the imbalance between the two canisters. This may be accomplished with a pressure sensor within or pneumatically coupled to each canister. Alternatively, the mass flow rate of oxygen enriched air being produced by each canister may be directly sensed, sampled, and converted to a difference value representative of the imbalance between the two canisters. This may be accomplished with a mass flow rate sensor within or pneumatically coupled to each canister. The representative difference value could then be used in an imbalance control system to dynamically adjust the duration of one or more phases of the PSA cycle in order to reduce the imbalance and optimise the overall yield of oxygen enriched air. This is the approach most often taken in industrial-scale PSA processes for gas separation involving multiple adsorbent containers.

However, such instrumentation of the PSA cycle is costly for small-scale portable oxygen concentrators designed for therapeutic use such as the oxygen concentrator 100. In addition, the pressure signal from the accumulator pressure sensor 107 might not be a sufficiently reliable guide to the pressure in each canister over the PSA cycle in the pneumatic architecture of FIG. 1B to be used for accurate imbalance control.

As mentioned above, the power parameter of the motor control signal 3030 in the motor control circuit 3000, while the compressor is being regulated to constant speed, is representative of (i.e. a sufficiently workable proxy for) the pressure within the canister currently connected to the compressor 210. During the fine pressure regulation mode described above, the motor speed may vary to regulate the system pressure to a target pressure value. However, within a PSA cycle the motor speed varies slowly compared to the variation in load 290 on the motor 220 and therefore the power parameter of the motor control signal 3030 being generated by the motor control circuit 3000. The power parameter of the motor control signal 3030 is therefore a sufficiently workable proxy for the pressure within the canister currently connected to the compressor 210. Thus, the motor control signal 3030 may be evaluated to reduce imbalance between the canisters of the POC.

One example of a power parameter is the duty cycle of the PWM waveform that is generated as the motor control signal 3030 by the motor controller 270 in one implementation of the present technology. FIG. 4 contains a graph 4000 containing the canister pressure waveforms 2050 and 2060 over a PSA cycle from FIG. 2, with the PWM duty cycle waveform 4010 over the same PSA cycle synchronously superimposed. FIG. 4 illustrates that the PWM duty cycle waveform 4010 closely tracks the shape of the pressure waveform of canister 302 over phases 1 to 4 when canister 302 is connected to the compressor 210 by the de-actuation of A-valve 122, and closely tracks the shape of the pressure waveform of canister 304 over phases 5 to 8 when canister 304 is connected to the compressor 210 by the de-actuation of B-valve 124.

In what follows, the power parameter is the duty cycle of the PWM waveform, but other implementations of the power parameter of the motor control signal 3030 are also contemplated.

The PWM duty cycle waveform may be determined from the PWM motor control signal 3030, or from the control parameter(s) that are derived by the compressor motor controller to generate such a signal in its regulation of the operation of the motor, such as when the compressor motor controller is implemented with or in software logic. The PWM duty cycle waveform may be sampled at a sampling interval that is short compared to the length of the PSA cycle, e.g. 5 ms or 10 ms. FIG. 4 also illustrates eight sampling points over the PSA cycle at which the PWM duty cycle waveform sample values may be recorded. The values at the eight recorded sampling points are labelled as SA1, SA2, SA3, SA4, SB1, SB2, SB3, and SB4 and represent samples of the PWM duty cycle waveform in relation to each of the eight phases of the PSA cycle. In one implementation, the sampling points coincide with the eight phase transitions, so the eight sample values may be defined as in Table 4:

TABLE 4
Definitions of PWM duty cycle sample values over a PSA cycle

| Sample | | |
|---|---|---|
| value | PWM Duty cycle | |
| label | value . . . | Proxy for: |
| SA1 | At start of phase 1 | Pressure in canister 302 at start |
| | | of its pressurisation phase (or |
| | | pressure in canister 304 at start |
| | | of its desorb/vent phase, |
| | | whichever is greater) |
| SA2 | At start of phase 2 | Pressure in canister 302 at start |
| | | of its adsorption phase |
| SA3 | At start of phase 3 | Pressure in canister 302 at start |
| | | of its equalisation phase (1) |
| SA4 | At start of phase 4 | Pressure in canister 302 at start |
| | | of its equalisation phase (2) |
| SB1 | At start of phase 5 | Pressure in canister 304 at start |
| | | of its pressurisation phase |
| | | (or pressure in canister 302 at |
| | | start of its desorb/vent phase, |
| | | whichever is greater) |
| SB2 | At start of phase 6 | Pressure in canister 304 at start |
| | | of its adsorption phase |
| SB3 | At start of phase 7 | Pressure in canister 304 at start |
| | | of its equalisation phase (1) |
| SB4 | At start of phase 8 | Pressure in canister 304 at start |
| | | of its equalisation phase (2) |

In one example implementation, each sample value represents an average of three sample values at sampling instants leading up to and including the corresponding sampling point.

For deriving a dynamic indication of a pneumatic imbalance, the sample(s) for any or each phase associated with one canister may then be compared with the sample(s) for a corresponding phase associated with the other canister(s). For example, the differences between SA1 and SB1, SA2 and SB2, SA3 and SB3, and SA4 and SB4 may be computed for each PSA cycle and labelled as SD1, SD2, SD3, and SD4 respectively. Likewise, the ratios between SA1 and SB1, SA2 and SB2, SA3 and SB3, and SA4 and SB4 may be computed for each PSA cycle and labelled as SR1, SR2, SR3, and SR4 respectively. Results from the comparisons, such as the values for the differences SD1, SD2, SD3, and SD4 and/or the ratios SR1, SR2, SR3, and SR4, may be obtained every PSA half cycle.

Each of the results (e.g., the computed duty cycle differences SD1, SD2, SD3, and SD4 or ratios SR1, SR2, SR3, and SR4) represents a measure of pressure imbalance between the canisters and may therefore be used as an input to an imbalance controller configured to regulate that measure of pressure imbalance to an imbalance target value, typically zero. For example, the imbalance controller may be implemented by a process or algorithm (e.g., a proportional—integral (PI) or proportional—integral—derivative (PID) control loop algorithm or control logic) of a processor of the controller 400 of the POC 100. The output of the imbalance controller is a dynamic adjustment to one or more phase durations of the PSA cycle that may be implemented as valve control signal timing adjustment(s). For example, Table 5 lists eight labels for dynamic phase duration adjustment values that may be generated by an imbalance controller according to the present technology, alongside what phase duration each adjustment value adjusts and what valve state change(s) is/are delayed by the adjustment. (Note a negative adjustment value causes a valve state change to be brought forward in time rather than delayed.)

TABLE 5
Definitions of dynamic phase duration
adjustment labels and their effects

| | | Valve state |
|---|---|---|
| | Dynamic adjustment | change(s) |
| Label | to duration of . . . | delayed |
| PA0 | Pressurisation phase of | G valve actuation |
| | canister 302 | |
| GA0 | Adsorption phase of | E-valve actuation |
| | canister 302 | |
| E1A | Equalisation phase (1) | B-valve de-actuation |
| | of canister 302 | |
| E2A | Equalisation phase (2) | G-valve de-actuation, |
| | of canister 302 | E-valve de-actuation, |
| | | A-valve actuation |
| PB0 | Pressurisation phase of | G-valve actuation |
| | canister 304 | |
| GB0 | Adsorption phase of | E-valve actuation |
| | canister 304 | |
| E1B | Equalisation phase (1) | A-valve de-actuation |
| | of canister 304 | |
| E2B | Equalisation phase (2) | G-valve de-actuation, |
| | of canister 304 | E-valve de-actuation, |
| | | B-valve actuation |

The dynamic adjustment values listed in Table 5 may be applied in real time to the phase durations on top of (in addition to) any static adjustment values to the phase durations such as those listed in Table 2.

FIG. 5 is a block diagram of an example of an imbalance control system 5000 for a POC, such as the POC 100, according to one implementation of the present technology. The imbalance control system 5000 may be implemented with control logic, such as for a processor of the POC or controller 400. The imbalance control system 5000 may have a predetermined imbalance target value, which may for example be set to zero. An optional adder 5010 of the imbalance control system 5000 may subtract a measure of imbalance from the imbalance target value (if the imbalance target value is not zero) to obtain an imbalance error. An imbalance controller 5020 converts the error into one or more dynamic phase duration adjustment values. For example, the imbalance controller 5020 may be implemented with a proportional-integral (PI) controller, which has two parameters, a proportional gain Kₚ and an integral gain K. The proportional gain Kₚ is multiplied by the imbalance error. The integral gain Kᵢ is multiplied by a sum of imbalance errors over some number of iterations (one iteration per PSA half cycle), and added to the result of multiplying the proportional gain by the imbalance error to obtain the dynamic phase duration adjustment. The dynamic phase duration adjustment is applied to the PSA state machine 5030 that controls the gas separation system 5040 of the POC (e.g., the gas separation system 110) to implement a PSA cycle as described above. A power parameter sampler 5050 samples the power parameter of the motor control signal (e.g., motor control signal 3030) that controls the motor of the gas separation system 5040 as described above in relation to FIG. 3, to generate a measure of imbalance every PSA half cycle as described above.

In general, an example instance of an imbalance control system 5000 may implement definitions for the following parameters: an imbalance target value, a measure of imbalance vector (choose n of SD1 to SD4 and SR1 to SR4), a dynamic phase duration adjustment vector (choose m of PAO, GAO, E1A, E2A, PB0, GBO, E1B, and E2B), an m by n proportional gain matrix Kₚ that is is multiplied by the imbalance error vector, and an m by n integral gain matrix Kᵢ that is multiplied by a sum of imbalance error vectors over some number of PSA half cycles (e.g. five).

Table 6 contains example definitions for parameters of an example imbalance control system 5000.

TABLE 6
Definitions for parameters of an example imbalance control system

| | | Dynamic | | |
|---|---|---|---|---|
| | Measure | phase | Imbalance | |
| | of | duration | controller | Imbalance |
| | imbalance | adjustment | proportional | controller |
| Imbalance | vector | vector | gain matrix | integral gain |
| target value | (n = 2) | (m = 2) | Kₚ | matrix Kᵢ |
| 0 | $[ \begin{matrix}SD 1 \\ SD 2\end{matrix} ]$ | $[ \begin{matrix}E 1 A \\ PB 0\end{matrix} ]$ | $[ \begin{matrix}100 & 0 \\ 0 & - 500\end{matrix} ]$ | $[ \begin{matrix}20 & 0 \\ 0 & - 100\end{matrix} ]$ |

Note the imbalance control system defined in Table 6, because of the diagonal nature of the matrices Kₚ and Kᵢ, effectively equates to two independent imbalance control systems operating simultaneously and in parallel, one taking SD1 as input and generating E1A values based on a Kₚ of 100 and a Kᵢ of 20, and one taking SD2 as input and generating PB0 values based on a Kₚ of −500 and a Kᵢ of −100.

In some implementations, rather than applying a negative value of a phase duration adjustment, the adjustment of a complementary phase duration is instead given a positive value of the same magnitude. For example, if the imbalance controller 5020 returns a negative value for E1A, a positive value of the same magnitude may be assigned to E2A, and E1A may be set to zero. Likewise, if the imbalance controller 5020 returns a negative value for PB0, a positive value of the same magnitude may be assigned to PAO, and PB0 may be set to zero. In such implementations, dynamic phase duration adjustments only ever lengthen phases of the PSA cycle, rather than shortening them.

FIG. 6 contains four graphs illustrating a potential effect of E1A adjustment on the imbalance between the PWM duty cycle waveforms of consecutive PSA half cycles in a POC supplying oxygen enriched air at setting 2 to patient with a breathing rate of 15 BPM. The two traces (A and B) in each graph represent the PWM duty cycle waveform over two consecutive PSA half cycles, averaged over multiple PSA cycles. In the top right of each graph is the oxygen purity of the output oxygen enriched air. The top left graph 6010, which corresponds to an E1A value of 0 (no adjustment) shows a significant imbalance between the two PWM duty cycle waveforms. The top right graph 6020, which corresponds to an E1A value of 10 ms, shows a slight reduction in the imbalance. The bottom left graph 6030, which corresponds to an E1A value of 20 ms, shows a further reduction in the imbalance. The bottom right graph 6040, which corresponds to an E1A value of 30 ms, shows almost complete balance between the two PWM duty cycle waveforms. It is notable that the oxygen purity increases from 84.7% to 86.4% to 87.4% to 89.4% as the adjustment value E1A increases from 0 to 30 ms, showing that controlling PWM imbalance materially affects the yield of oxygen enriched air.

In some implementations, the time series of differences SD1 to SD4 or ratios SR1 to SR4 may be low-pass filtered before being used in a measure of imbalance vector. The low-pass filter time constant may be long enough to comprise several PSA cycles.

In some implementations, the PWM duty cycle waveform over each PSA half cycle may be integrated. The difference between, or ratio of, the integrals of duty cycle over each PSA half cycle may be used as a measure of imbalance to be converted to a dynamic phase duration adjustment by the imbalance controller 5020 of the imbalance control system 5000.

### Glossary

For the purposes of the present technology disclosure, in certain forms of the present technology, one or more of the following definitions may apply. In other forms of the present technology, alternative definitions may apply.

### General

Air: In certain forms of the present technology, air may be taken to mean atmospheric air, consisting of 78% nitrogen (N₂), 21% oxygen (O₂), and 1% water vapour, carbon dioxide (CO₂), argon (Ar), and other trace gases.

Oxygen enriched air: Air with a concentration of oxygen greater than that of atmospheric air (21%), for example at least about 50% oxygen, at least about 60% oxygen, at least about 70% oxygen, at least about 80% oxygen, at least about 87% oxygen, at least about 90% oxygen, at least about 95% oxygen, at least about 98% oxygen, or at least about 99% oxygen. “Oxygen enriched air” is sometimes shortened to “oxygen”.

Medical Oxygen: Medical oxygen is defined as oxygen enriched air with an oxygen concentration of 80% or greater.

Ambient: In certain forms of the present technology, the term ambient will be taken to mean (i) external of the treatment system or patient, and (ii) immediately surrounding the treatment system or patient.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

Flow therapy: Respiratory therapy comprising the delivery of a flow of air to an entrance to the airways at a controlled flow rate referred to as the treatment flow rate that is typically positive throughout the patient's breathing cycle.

Patient: A person, whether or not they are suffering from a respiratory disorder.

Pressure: Force per unit area. Pressure may be expressed in a range of units, including cmH₂O, g-f/cm², pounds per square inch (psi), and hectopascals. 1 cmH₂O is equal to 1 g-f/cm² and is approximately 0.98 hectopascal (1 hectopascal=100 Pa=100 N/m²=1 millibar ˜0.001 atm ˜0.015 psi). Unless otherwise stated, in the present specification pressure values are given as gauge pressures (pressures relative to ambient atmospheric pressure).

### General Remarks

The term “coupled” as used herein means either a direct connection or an indirect connection (e.g., one or more intervening connections) between one or more objects or components. The phrase “connected” means a direct connection between objects or components such that the objects or components are connected directly to each other. As used herein the phrase “obtaining” a device means that the device is either purchased or constructed.

In the present disclosure, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative implementations of various aspects of the present technology may be apparent to those skilled in the art in view of this description. Accordingly, this description is to be construed as illustrative only and is for the purpose of teaching those skilled in the art the general manner of carrying out the technology. It is to be understood that the forms of the technology shown and described herein are to be taken as implementations. Elements and materials may be substituted for those illustrated and described herein, parts and processes may be reversed, and certain features of the technology may be utilized independently, all as would be apparent to one skilled in the art after having the benefit of this description of the technology. Changes may be made in the elements described herein without departing from the spirit and scope of the technology as described in the appended claims.

Label list

| | |
|---|---|
| oxygen concentrator | 100 |
| inlet | 101 |
| inlet | 105 |
| accumulator | 106 |
| pressure sensor | 107 |
| inlet muffler | 108 |
| gas separation system | 110 |
| inlet valve | 122 |
| inlet valve | 124 |
| filter | 129 |
| outlet | 130 |
| muffler | 133 |
| baffle | 139 |
| check valve | 142 |
| flow restrictor | 143 |
| check valve | 144 |
| outlet system | 150 |
| valve | 152 |
| flow restrictor | 153 |
| valve | 154 |
| flow restrictor | 155 |
| supply valve | 160 |
| oxygen sensor | 165 |
| housing | 170 |
| fan | 172 |
| outlet | 173 |
| outlet port | 174 |
| flow restrictor | 175 |
| power supply | 180 |
| particulate filter | 187 |
| connector | 190 |
| delivery conduit | 192 |
| pressure sensor | 194 |
| airway delivery device | 196 |
| mouthpiece | 198 |
| compression system | 200 |
| motor speed transducer | 201 |
| compressor | 210 |
| compressor outlet | 212 |
| motor | 220 |
| external armature | 230 |
| air transfer device | 240 |
| compressor outlet conduit | 250 |
| motor controller | 270 |
| motor driver | 280 |
| load | 290 |
| canister system | 300 |
| canister | 302 |
| canister | 304 |
| air inlet | 306 |
| housing component | 310 |
| base | 315 |
| valve seat | 322 |
| opening | 323 |
| valve seat | 324 |
| outlet | 325 |
| exhaust gas | 327 |
| inlet conduit | 330 |
| valve seat | 332 |
| valve seat | 334 |
| aperture | 337 |
| conduit | 342 |
| conduit | 344 |
| conduit | 346 |
| opening | 375 |
| controller | 400 |
| processor | 410 |
| internal memory | 420 |
| transceiver | 430 |
| housing component | 510 |
| conduit | 530 |
| conduit | 532 |
| opening | 542 |
| opening | 544 |
| valve seat | 552 |
| valve seat | 554 |
| control panel | 600 |
| input port | 605 |
| power button | 610 |
| flow rate setting button | 620 |
| flow rate setting button | 622 |
| flow rate setting button | 624 |
| flow rate setting button | 626 |
| active mode button | 630 |
| sleep mode button | 635 |
| altitude button | 640 |
| battery check button | 650 |
| LED | 655 |
| PSA cycle | 2000 |
| valve actuation waveform | 2010 |
| valve actuation waveform | 2020 |
| valve actuation waveform | 2030 |
| pressure waveform | 2050 |
| pressure waveform | 2060 |
| motor control circuit | 3000 |
| speed command | 3010 |
| speed signal | 3020 |
| motor control signal | 3030 |
| motor drive signals | 3040 |
| graph | 4000 |
| PWM duty cycle waveform | 4010 |
| imbalance control system | 5000 |
| adder | 5010 |
| imbalance controller | 5020 |
| PSA state machine | 5030 |
| gas separation system | 5040 |
| power parameter sampler | 5050 |
| graph | 6010 |
| graph | 6020 |
| graph | 6020 |
| graph | 6030 |

1. An oxygen concentrator comprising:
a compressor configured to generate a pressurised air stream;
at least two canisters, each canister comprising adsorbent material configured to preferentially adsorb a component gas from the pressurised air stream, thereby producing oxygen enriched air from the pressurised air stream;
one or more valves configured to:
selectively pneumatically couple the compressor to each canister so as to selectively feed the pressurised air stream to the canister; and
selectively vent each canister to atmosphere;
an accumulator pneumatically coupled to receive the produced oxygen enriched air;
one or more controllers operably coupled to the one or more valves and the compressor, the one or more controllers configured to:
regulate a speed of the compressor to a speed set point while generating the pressurised air stream, wherein the regulating comprises generating a compressor control signal having a characteristic parameter;
selectively operate the one or more valves in a cyclic pattern so as to produce oxygen enriched air in the accumulator, wherein a cycle of the cyclic pattern comprises a plurality of phases, each of the plurality of phases comprising a duration; and
generate a dynamic adjustment to one or more of the durations based on an evaluation of the characteristic parameter, whereby the dynamic adjustment reduces dynamic imbalance of a pneumatic characteristic between the canisters.

2. The oxygen concentrator of claim 1, wherein the one or more controllers comprises an imbalance control system configured to generate the dynamic adjustment to the one or more durations, wherein the imbalance control system comprises:
a sampler configured to:
sample one or more values of the characteristic parameter over a cycle, and
compute a measure of imbalance based on the sampled values; and
an imbalance controller configured to compute at least one phase duration adjustment from the measure of imbalance.

3. The oxygen concentrator of claim 2, wherein the imbalance controller is configured to compute the at least one phase duration adjustment based on a comparison between the measure of imbalance and an imbalance target value.

4. The oxygen concentrator of claim 3 wherein the comparison comprises a difference between the measure of imbalance and an imbalance target value.

5. The oxygen concentrator of any one of claims 2 to 4, wherein the imbalance controller is a proportional-integral-derivative (PID) or proportional-integral (PI) controller.

6. The oxygen concentrator of any one of claims 2 to 5, wherein the sampler is configured to compute the measure of imbalance as a vector comprising one or more of:
one or more differences between sample values at respective sampling points of consecutive half cycles; and
one or more ratios between sample values at respective sampling points of consecutive half cycles.

7. The oxygen concentrator of claim 6, wherein the sampling points coincide with phase transitions of the cycle.

8. The oxygen concentrator of any one of claims 2 to 7, wherein the sampler is further configured to compute each sample value at a sampling point from a plurality of sample values leading up to and including the sampling point.

9. The oxygen concentrator of any one of claims 1 to 2 wherein the evaluation comprises a comparison between (a) a first sample value of the characteristic parameter, the first sample value being associated with at least one first phase for one of the at least two canisters, and (b) a second sample value of the characteristic parameter, the second sample value being associated with at least one second phase for another one of the at least two canisters, wherein the at least one first phase and the at least one second phase are corresponding phases.

10. The oxygen concentrator of claim 9 wherein the comparison comprises a difference between the first sample value and the second sample value.

11. The oxygen concentrator of claim 9 wherein the comparison comprises a ratio of the first sample value and the second sample value.

12. The oxygen concentrator of any one of claims 9 to 11 wherein the evaluation further comprises determination of an error based on the comparison.

13. The oxygen concentrator of claim 12 wherein the error is determined from a target imbalance value.

14. The oxygen concentrator of any one of claims 12 to 13 wherein the evaluation comprises inputting the error to a proportional-integral-derivative (PID) or proportional-integral (PI) controller configured to generate the dynamic adjustment of the one or more of the durations.

15. The oxygen concentrator of any one of claims 1 to 14, wherein to regulate speed of the compressor to a speed set point, the one or more controllers are configured to generate the compressor control signal based on a difference between (a) a measured speed signal generated by a speed sensor, and (b) the speed set point.

16. The oxygen concentrator of any one of claims 1 to 15, wherein the compressor control signal is a pulse width modulation (PWM) waveform and the characteristic parameter is a duty cycle of the PWM waveform.

17. A method of operating an oxygen concentration device, the method comprising:
controlling, with one or more controllers, a compressor to generate a pressurised air stream to at least two canisters, each canister comprising adsorbent material configured to preferentially adsorb a component gas from the pressurised air stream, thereby producing oxygen enriched air from the pressurised air stream to an accumulator pneumatically coupled to receive the produced oxygen enriched air; the controlling the compressor comprising regulating a speed of the compressor to a speed set point, wherein the regulating comprises generating a compressor control signal having a characteristic parameter;
controlling, with the one or more controllers, operation of one or more valves to (a) selectively pneumatically couple the compressor to each canister so as to selectively feed the pressurised air stream to the canister, and (b) selectively vent each canister to atmosphere;
wherein the controlling operation of the one or more valves comprises selectively operating the one or more valves in a cyclic pattern so as to produce the oxygen enriched air, wherein a cycle of the cyclic pattern comprises a plurality of phases, each of the plurality of phases comprising a duration; and
controlling, with the one or more controllers, generation of a dynamic adjustment to one or more of the durations based on an evaluation of the characteristic parameter, whereby the dynamic adjustment reduces dynamic imbalance of a pneumatic characteristic between the canisters.

18. The method of claim 17, wherein to generate the dynamic adjustment to the one or more durations, the one or more controllers:
samples one or more values of the characteristic parameter over a cycle, and
computes a measure of imbalance based on the sampled values; and
compute at least one phase duration adjustment from the measure of imbalance.

19. The method of claim 18, wherein to compute the at least one phase duration adjustment, the one or more controllers compares the measure of imbalance and an imbalance target value.

20. The method of claim 19 wherein to compare the measure of imbalance and an imbalance target value, the one or more controllers computes a difference between the measure of imbalance and an imbalance target value.

21. The method of any one of claims 18 to 20, wherein the one or more controllers applies proportional-integral-derivative (PID) control or proportional-integral (PI) control to the computed difference.

22. The method of any one of claims 18 to 21, wherein the one or more controllers computes the measure of imbalance as a vector comprising one or more of:
one or more differences between sample values at respective sampling points of consecutive half cycles; and
one or more ratios between sample values at respective sampling points of consecutive half cycles.

23. The method of claim 22, wherein the sampling points coincide with phase transitions of the cycle.

24. The method of any one of claims 18 to 22, wherein the one or more controllers computes each sample value at a sampling point from a plurality of sample values leading up to and including the sampling point.

25. The method of any one of claims 17 to 24, wherein the evaluation comprises comparing (a) a first sample value of the characteristic parameter, the first sample value being associated with at least one first phase for one of the at least two canisters, and (b) a second sample value of the characteristic parameter, the second sample value being associated with at least one second phase for another one of the at least two canisters, wherein the at least one first phase and the at least one second phase are corresponding phases.

26. The method of claim 25 wherein the comparing comprises computing a difference between the first sample value and the second sample value.

27. The method of claim 25 wherein the comparing comprises computing a ratio of the first sample value and the second sample value.

28. The method of claim 27 wherein the evaluation further comprises determining an error based on the comparing.

29. The method of claim 28 wherein the error comprises is determined from a target imbalance value.

30. The method of any one of claims 28 to 29 wherein the evaluation comprises inputting the error to a proportion-integral-derivative (PID) or proportional-integral (PI) controller to generate the dynamic adjustment of the one or more of the durations.

31. The method of any one of claims 17 to 30, wherein the regulating a speed of the compressor to a speed set point comprises generating the compressor control signal based on a difference between (a) a measured speed signal generated by a speed sensor, and (b) the speed set point.

32. The method of any one of claims 17 to 31, wherein the compressor control signal is a pulse width modulation (PWM) waveform and the characteristic parameter is a duty cycle of the PWM waveform.

33. Apparatus comprising:
means for controlling a compressor to generate a pressurised air stream to at least two canisters, each canister comprising adsorbent material configured to preferentially adsorb a component gas from the pressurised air stream, thereby producing oxygen enriched air from the pressurised air stream to an accumulator pneumatically coupled to receive the produced oxygen enriched air; the controlling the compressor comprising regulating a speed of the compressor to a speed set point, wherein the regulating comprises generating a compressor control signal having a characteristic parameter;
means for controlling operation of one or more valves to (a) selectively pneumatically couple the compressor to each canister so as to selectively feed the pressurised air stream to the canister, and (b) selectively vent each canister to atmosphere; wherein the controlling operation of the one or more valves comprises selectively operating the one or more valves in a cyclic pattern so as to produce the oxygen enriched air, wherein a cycle of the cyclic pattern comprises a plurality of phases, each of the plurality of phases comprising a duration; and
means for controlling generation of a dynamic adjustment to one or more of the durations based on an evaluation of the characteristic parameter, whereby the dynamic adjustment reduces dynamic imbalance of a pneumatic characteristic between the canisters.
